(19) United States
(12) Patent Application Publication
(10) Pub. No.: US 2022/0213462 A1
(43) Pub. Date: Jul. 7, 2022

(54) RED BLOOD CELLS EXPRESSING VON WILLEBRAND FACTOR PROTEASE AND METHODS OF USE THEREOF

(71) Applicant: Albert Einstein College of Medicine, Bronx, NY (US)

(72) Inventors: Eric Bouhassira, Hastings On Hudson, NY (US); Khulan Batbayar, Pelham, NY (US); Karl Roberts, Brooklyn, NY (US)

(73) Assignee: Albert Einstein College of Medicine, Bronx, NY (US)

(21) Appl. No.: 17/605,864

(22) PCT Filed: Apr. 17, 2020
(86) PCT No.: PCT/US2020/028736

§ 371 Date: Oct. 22, 2021

Related U.S. Application Data
(60) Provisional application No. 62/839,065, filed on Apr. 26, 2019.

(51) Int. Cl. C12N 9/64; A61K 35/18; C12N 5/078; A61P 7/02; C07K 14/745

(52) U.S. Cl. CPC C12N 9/6489; C12Y 304/24087; A61K 35/18; C12N 5/0641; A61K 38/00; C07K 14/745; C07K 2319/035; C12N 2510/00; C12N 2506/45; A61P 7/02

(57) ABSTRACT

This disclosure provides methods and compositions for treating TTP based on transfusion of a relatively small number of genetically modified red blood cells. The genetically modified red blood cells express a fusion protein including a fragment of ADAMTS13 that is enzymatically active against von Willebrand factor (VWF). The fragments of ADAMTS13 can be resistant to the inhibitors, e.g., the auto-immune antibodies, which are responsible for the acquired form of TTP.

### CROSS-REFERENCE TO RELATED APPLICATION

This application claims the benefit of U.S. Provisional Patent Application No. 62/839,065, filed Apr. 26, 2019, the contents of which is herein incorporated by reference in its entirety.

### STATEMENT REGARDING FEDERALLY SPONSORED RESEARCH

This invention was made with government support under R01HL130764 awarded by National Institutes of Health. The government has certain rights in the invention.

### FIELD OF THE INVENTION

The present invention relates generally to red blood cells expressing von Willebrand factor (VWF) protease and more specifically to red blood cells expressing ADAMTS13 and methods of using the same.

### BACKGROUND OF THE INVENTION

Thrombotic Thrombocytopenic Purpura (TTP) is a disorder of the blood that can be clinically diagnosed by the presence of micro-angiopathic hemolytic anemia, schistocytes, and thrombocytopenia in the absence of other likely etiologies. The symptoms associated with this disease include chest pain, headaches, confusion, speech changes, and alterations in consciousness, which vary from lethargy to coma; other symptoms include development of kidney abnormalities. These symptoms can be very severe, and fatal. The disease strikes about 4 out of every 100,000 people. It is seen most commonly in adults from 20 to 50 years old, with women affected slightly more often than men. In most TTP patients, the onset of the disease occurs in otherwise healthy individuals, and there is no history of a similar condition in other family members. However, in a smaller set of individuals, there is evidence suggesting that the condition may be inherited.

It has been demonstrated that TTP is associated with the presence of ultra-large von Willebrand factor (VWF) multimers that was caused by the deficiency of a plasma factor, which was later identified as ADAMTS13. Positional cloning confirmed that ADAMTS13 is also responsible for the congenital form of TTP. Some of the molecular aspects of TTP are now well understood. Low ADAMTS13 activity decreases the normal cleavage rate of VWF, which then accumulates in its high molecular weight form. These high molecular weight VWF molecules unfold in the presence of shear stress in the circulation and interact with the vessel walls and platelets, promoting thrombi formation in the absence of injury, which can lead to life-threatening microvascular thrombosis and the clinical manifestations of TTP. The idiopathic form of TTP has an incidence of about 1/250,000 per year and is caused by auto-antibodies that inactivate ADAMTS13. Anti-ADAMTS13 antibodies are mostly IgG4 and IgG1 and can either inhibit the proteolytic activity, enhance the clearance, or disturb the interaction with physiologic binding partners of ADAMTS13.

The current treatment for idiopathic TTP relies on plasma exchange requiring infusion of several liters of concentrate for up to several weeks. Plasma exchange, complemented or not with rituximab, an anti-CD20 antibody that suppresses the production of autoantibodies, or with caplacizumab, a nanobody of VWF that blocks VWF-platelet aggregation, is a life-saving but cumbersome procedure that has significant toxicity, a high number of relapses, and a 10-20% rate of mortality. Congenital TTP represents about 5% of all TTP cases. Congenital TTP is treated in a similar manner as the idiopathic form but with lower doses of plasma.

Recombinant ADAMTS13 is currently being tested to treat congenital TTP. This approach could also potentially be used to treat the idiopathic form, but in the absence of antibody resistant forms of recombinant ADAMTS13 with long half-lives, infusion of a large amount of the protein will be required in order to saturate the auto-antibodies, since plasma exchange works in large part by removing the auto-antibodies.

Thus, there remains a strong need for developing improved methods and reagents to treat the disease, to decrease fatality, and to decrease the severity of the symptoms associated with the disease.

### SUMMARY OF THE INVENTION

This disclosure addresses the need mentioned above in a number of aspects. In one aspect, this disclosure provides a genetically modified red blood cell. The red blood cell is engineered to express on the surface thereof a fusion protein comprising a fragment of ADAMTS13 that is enzymatically active against von Willebrand factor (VWF). The fragment of ADAMTS13 may have an amino acid sequence at least 75% identical to the sequence of SEQ ID NOs: 1, 2, 3, 9, 10, 11, 12, or 13.

In some embodiments, the fusion protein comprises a lipid anchor operably linked to the fragment of ADAMTS13. For example, the lipid anchor can be operably linked to the C-terminal end of the fragment of ADAMTS13. The lipid anchor can be a Glycosylphosphatidylinositol (GPI) anchor. The GPI anchor may include the amino acid sequence of SEQ ID NO: 4.

In some embodiments, the red blood cell is transduced with a retrovirus comprising a nucleic acid encoding the fusion protein. The nucleic acid may include a nucleotide sequence at least 75% identical to a nucleic acid sequence of SEQ ID NOs: 5, 6, or 7.

In another aspect, a method for treating thrombotic thrombocytopenic purpura (TTP) is also provided. The method includes administering a therapeutically effective amount of the genetically modified red blood cells as described above to a subject in need thereof. TTP may include hereditary TTP, congenital TTP, acquired TTP, or immune-mediated TTP.

The red blood cells can be administered by infusion. In some embodiments, the method may include producing the red blood cells in vitro before administrating to the subject. In some embodiments, the red blood cells can be produced in a hollow fiber culturing system by expansion of hematopoietic progenitors.

In some embodiments, this disclosure also provides a method for preparing the above-described red blood cells. The method includes: (i) providing a plurality of stem cells; (ii) contacting the stem cells with a nucleic acid encoding a fusion protein comprising ADAMTS13 or fragment thereof to obtain transduced stem cells; (iii) expanding the transduced stem cells cells in cell culture medium; and (iv) collecting the resulting red blood cells.

Also within the scope of this disclosure is a composition comprising the above-described stem cells and/or red blood cells and optionally a cryo-protectant. In another aspect, this disclosure also provides blood, cellular and acellular blood components, or blood products obtained from the red blood cell as described above.

In another aspect, a method for increasing the level of functional ADAMTS13 in a subject is provided. The method includes administering an effective amount of the red blood cells as described above to the subject.

In yet another aspect, a method for decreasing aggregation of VWR in a subject is provided. The method includes administering an effective amount of the red blood cells as described herein to the subject.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a diagram showing a structure of ADAMTS13. The top diagram depicts the domain structure of ADAMTS13 and the mutation in ADAMTS13 that confer resistance to TTP inhibitors. AD2-5 are four truncated fragments studied that display ADAMTS13 activity but that differ by their sensitivity (AD5) or resistance to TTP inhibitors. AD5-RES contains 5 mutations as indicated.

FIGS. 2A, 2B, 2C, and 2D (collectively “FIG. 2”) are a set of diagrams showing a pluripotent stem cell-robust erythroid differentiation protocol (PSC-RED). FIG. 2A is the graph showing the long PSC-RED protocol used to differentiated GPI-ADAMTS13-iPSCs into red blood cells. The stages of differentiation induced by successive incubation in supplements S1, S2, S3, S4, SED SER, and SER2 (see methods) include mesoderm induction, hematopoietic progenitor cell (HPC) specification and expansion, and erythroid specification, expansion, maturation, and enucleation. The base culture media are E8 prior to iPSC differentiation, IMIT from day 0 to day 24, and R6 from day 24 to 45. Bottom rows: S1-S4, SED, SER, SER2: Small molecules and cytokine supplements that are necessary to differentiate iPSCs into cRBCs. IMIT and R6: two chemically defined albumin-free media developed by the inventors. FIG. 2B shows the yield of PSC-RED as compared to the best previously published protocol. One iPSC yields more than 100,000 erythroid cells. FIG. 2C shows FACS analysis demonstrating about 50% enucleation. Draq5: nuclear stain. FIG. 2D shows Giemsa staining after filtration of nuclei and orthochromatic erythroblasts demonstrating production of a 99% pure batch of enucleated cRBCs from iPSCs.

FIGS. 3A and 3B (collectively “FIG. 3”) show a construct to insert GPI-ADAMTS13 at the AAVS1 safe harbor site. FIG. 3A is a graph showing a DNA construct that can be used to genetically modified cells to express GPI-ADAMTS13 in their membrane. The construct contains arms (left arm and right arm) that are homologous to sequences within the first intron of gene PPP1R12C also known as safe harbor AAVS1. The construct also contains a splice acceptor and the coding sequence of the puromycin genes to facilitate selection of clones that have been successfully genetically modified. In addition, the construct contains a cis-acting regulatory element from the human alpha and beta-globin gene cluster. Specifically, the construct contains the beta-globin mini LCR and the promoter of the alpha1 globin gene which confers high-level of erythroid-specific expression. It also contains the beginning of the first intron of the alpha globin 2 genes which is fused to the cDNA for the first 745 amino acids of human ADAMTS13 itself fused to the DAF GPI anchor sequence. The cDNA is interrupted by intron1 of the human beta-globin gene and ends with the 3′UTR and polyA adenylation signal from the human beta-globin genes to improve expression. FIG. 3B is a graph showing the theoretical structure of the GPI-ADAMTS13 construct inserted into a cell membrane.

FIGS. 4A and 4B (collectively “FIG. 4”) are a set of diagrams showing the methods to insert GPI-ADAMTS13 at the AAVS1 safe harbor site using a CRISPR-Cas9 system. FIG. 4A shows an RNP complex containing guide RNA to target the AAVS1 locus complexed to recombinant cas9 protein (Figure adapted from https://cellculturedish.com/the-crispr-cas9-system-and-its-applications/). FIG. 4B shows the pAD5 plasmid which is the vector carrying the targeting construct described in FIG. 3A.

FIGS. 5A and 5B (collectively “FIG. 5”) show a demonstration of insertion of AD5 construct at AAVS1 safe harbor in K562 cells. FIG. 5A is a graph showing the location of primers P1 and P4 within the Chr19: PPP1R12C region and of primer P3 in the AD5 construct of FIG. 4B. FIG. 5B is the micrographs showing the results of the analysis of the resulting PCR fragments by agarose gel electrophoresis. Specifically, 100 ng of genomic DNA from two puromycin resistant K562 clones obtained after transfection of plasmid AD5 and the appropriate sgRNA/cas9 complex was mixed with either primer pair P1/P2 (top micrograph) or with primer P1/P4 (bottom micrograph) and amplified by PCR for 30 cycles. The leftmost lane is a size marker, the second lane is a no DNA control to test for contamination. Lanes 3 and 4 illustrate that a fragment of the appropriate size was obtained with primer P1 and P2 in both clones tested, but only in one of the two clones with primer pair P1/P4. The presence of a PCR product with primer pair P1/P2 and the absence of a band with primer pair P1/P4 indicate a homozygous insertion (hom). The presence of a PCR product of the appropriate size with bot primer pairs indicates a heterozygous insertion (Het).

FIGS. 6A and 6B (collectively “FIG. 6”) show a demonstration of insertion of AD5 construct at AAVS1 safe harbor in human iPSCs. FIG. 6A is a graph showing a PCR analysis similar to that of FIG. 5B but for puromycin resistant iPSC clones obtained as in FIG. 5B. Two heterozygous and one homozygous clones are shown. FIG. 6B is a micrograph showing the morphology of an undifferentiated iPSC clone as observed by phase contrast microscopy.

FIG. 7 shows expression of GPI-ADAMTS13 on the membrane of K562 cells. Two K562 clones containing construct AD5 inserted at AAVS1, as demonstrated in FIGS. 5A and 5B, were stained with a FITC-labelled antibody against ADAMTS13 and analyzed by flow cytometry. The Dotplot on the left illustrates the FSC-H and SSC-H pattern of the two GPI-ADAMTS12 K562 clones. The histograms on the right illustrate the fluorescence in the FITC channel demonstrating expression of GPI-ADAMTS13 in almost all cells. The red and blue histograms respectively illustrate the fluorescence observed with K562 cells containing GPI-ADAMTS13 or with control untransfected cells. The middle Dotplot shows the same FITC fluorescence but as a function of forward scatter (FSC-H).

FIGS. 8A and 8B (collectively “FIG. 8”) show a demonstration of ADAMTS13 enzymatic activity of K562 cells expressing GPI-ADAMTS13 on their membrane. FIG. 8A is a graph showing the VWF73 FRET assay used to detect ADAMTS13 enzymatic activity. The assay is based on cleavage of peptide VWF73 which encompasses the VWF factor ADAMTS13 cognate recognition site. The peptide has been modified to comprise two fluorophores (Nma and Dnp) which interfere with each other because of their close proximity. When the peptide is cleaved the interference is relieved and the fluorescence emitted is proportional to ADAMTS13 activity. FIG. 8B shows X-Y scatter plots illustrating the ADAMTS13 enzymatic activity of the two GPI-ADAMTS13 K562 clones. Peptide VWF73 was incubated either with 0.4, 0.8 or 1.2 μL of normal human plasma, with 200,000 GPI-ADAMTS13 K562 cells or with untransfected control K562 cells. Fluorescence was then measured over time on a cytofluor II fluorimeter. Analysis of the results suggested that 200,000 GPI-ADAMTS13 K562 cells from clone 3 had about the same activity GPI-ADAMTS13 as 10 μL of plasma (n=3). This suggests that 5 mL of GPI-ADAMTS13 red blood cells (2.5×10¹⁰ cells) would be equivalent to 1 liter of plasma.

FIGS. 9A and 9B (collectively “FIG. 9”) show analysis of GPI-ADAMTS13 expression and of ADAMTS13 enzymatic activity of erythroid cells produced by differentiation of GPI-ADAMTS13 iPSCs. FIG. 9A shows GPI-ADAMTS13 iPSCs were differentiated using the PSC-RED long protocol depicted in FIG. 2, and analyzed for ADAMTS13 expression as described in FIG. 7. Erythroid cells produced by differentiation of GPI-ADAMTS13 iPSC clones express GPI-ADAMTS13 in most cells. FIG. 9B shows the VWF73 FRET assay, as described in FIGS. 8A and 8B, was used to test the enzymatic activity of erythroid cells produced by differentiation of GPI-ADAMTS13 iPSCs. 200,000 erythroid cells were compared to 0.4, 0.8 or 1.6 μL of normal human plasma. 200,000 GPI-ADAMTS13 erythroid cells derived from iPSCs were about as active as 10 μL of plasma (n=3).

FIG. 10 shows antibody resistant fragment AD2, AD3, and AD4 of ADAMTS13. cDNA fragments coding for variants AD2, AD3, and AD4 were cloned instead of fragment AD5 in plasmid pAD5 described in FIGS. 4A and 4B, creating plasmid pAD2, pAD3, and pAD4.

FIG. 11 shows expression of GPI-AD2, GPI-AD3, and GPI-AD4 on the membrane of K562 cells. Plasmids pAD2, pAD3, and pAD4 were inserted at the AAVS1 site in K562 as described in FIGS. 5A and 5B and expression of fragments GPI-AD2, GPI-AD3 and GPI-AD4 assessed as described in FIG. 7 demonstrating a high level of expression of this ADAMTS13 fragment on the membrane of these cells.

FIG. 12 show analysis of the enzymatic activity of GPI-AD2, GPI-AD3, and GPI-AD4. 200,000 K562 cells containing pAD2, pAD3, and pAD4 inserted at the AAVS1 site, and control untransfected K562 cells were compared to 0.4 and 0.8 μL of normal human plasma using the VWF73 FRET assay. This revealed that all three fragments were enzymatically active.

### DETAILED DESCRIPTION OF THE INVENTION

This disclosure provides a new method for treating TTP based on transfusion of a relatively small number of genetically modified red blood cells. The genetically modified red blood cells, also termed ADAMTS13-RBCs, express a fusion protein including a fragment of ADAMTS13 that is enzymatically active against von Willebrand factor (VWF). The fragment of ADAMTS13 can be resistant to the inhibitors, e.g., the auto-antibodies, which are responsible for the acquired form of TTP.

This disclosure demonstrates that the fusion protein can be expressed at very high levels and that the membrane-bound ADAMTS13 is enzymatically active against VWF. Comparison of enzymatic activity with plasma concentrate indicates that about 5×10¹⁰ of ADMTS13-RBCs would be sufficient to deliver an amount of ADAMTS13 equivalent to 2 liters of plasma. This indicates that a transfusion of about 10 mL of ADAMTS13-RBCs could be therapeutic for congenital and acquired TTP.

The advantages of the disclosed red blood cells include: (1) the half-life of the membrane-bound ADAMTS13 in the circulation can be much longer than that of ADAMTS13 injected as a recombinant form or as part of plasma concentrate; and (2) the need of multiple injection is reduced when red blood cells express an inhibitor-resistant form of ADAMTS13.

### I. RED BLOOD CELLS EXPRESSING ADAMTS13 OR A FRAGMENT THEREOF

In one aspect, this disclosure provides a genetically modified red blood cell. The red blood cell is engineered to express on the surface thereof a fusion protein comprising a fragment of an ADAMTS13 protein or a variant thereof that is enzymatically active against VWF.

Also within the scope of this disclosure are the variants and homologs with significant identity to ADAMTS13. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of ADAMTS13 described herein.

In some embodiments, the fragment of the ADAMVTS13 protein or variant thereof may have an amino acid sequence at least 75% identical to the sequence of SEQ TD NOs. 1, 2, 3, 8, 9, 10, 11, 12, or 13.

TABLE 1
Sequence table

| | | OTHER |
|---|---|---|
| SEQ ID NO | SEQUENCES | INFORMATION |
| SEQ ID NO: | MHQRHPRARCPPLCVAGILACGFLLGCWGPSHFQQSCL | ADAMTS13 |
| 1 | QALEPQAVSSYLSPGAPLKGRPPSPGFQRQRQRQRRAA | (full length; |
| | GGILHLELLVAVGPDVFQAHQEDTERYVLTNLNIGAEL | 1-1427 aa) |
| | LRDPSLGAQFRVHLVKMVILTEPEGAPNITANLTSSLLS | NP_620594 |
| | VCGWSQTINPEDDTDPGHADLVLYITRFDLELPDGNRQ | (ADAMTS1 |
| | VRGVTQLGGACSPTWSCLITEDTGFDLGVTIAHEIGHSF | 3 isoform 1) |
| | GLEHDGAPGSGCGPSGHVMASDGAAPRAGLAWSPCSR | |
| | RQLLSLLSAGRARCVWDPPRPQPGSAGHPPDAQPGLYY | |
| | SANEQCRVAFGPKAVACTFAREHLDMCQALSCHTDPL | |
| | DQSSCSRLLVPLLDGTECGVEKWCSKGRCRSLVELTPIA | |
| | AVHGRWSSWGPRSPCSRSCGGGVVTRRRQCNNPRPAF | |
| | GGRACVGADLQAEMCNTQACEKTQLEFMSQQCARTD | |
| | GQPLRSSPGGASFYHWGAAVPHSQGDALCRHMCRAIG | |
| | ESFIMKRGDSFLDGTRCMPSGPREDGTLSLCVSGSCRTF | |
| | GCDGRMDSQQVWDRCQVCGGDNSTCSPRKGSFTAGR | |
| | AREYVTFLTVTPNLTSVYIANHRPLFTHLAVRIGGRYVV | |
| | AGKMSISPNTTYPSLLEDGRVEYRVALTEDRLPRLEEIRI | |
| | WGPLQEDADIQVYRRYGEEYGNLTRPDITFTYFQPKPR | |
| | QAWVWAAVRGPCSVSCGAGLRWVNYSCLDQARKELV | |
| | ETVQCQGSQQPPAWPEACVLEPCPPYWAVGDFGPCSAS | |
| | CGGGLRERPVRCVEAQGSLLKTLPPARCRAGAQQPAV | |
| | ALETCNPQPCPARWEVSEPSSCTSAGGAGLALENETCV | |
| | PGADGLEAPVTEGPGSVDEKLPAPEPCVGMSCPPGWGH | |
| | LDATSAGEKAPSPWGSIRTGAQAAHVWTPAAGSCSVSC | |
| | GRGLMELRFLCMDSALRVPVQEELCGLASKPGSRREVC | |
| | QAVPCPARWQYKLAACSVSCGRGVVRRILYCARAHGE | |
| | DDGEEILLDTQCQGLPRPEPQEACSLEPCPPRWKVMSLG | |
| | PCSASCGLGTARRSVACVQLDQGQDVEVDEAACAALV | |
| | RPEASVPCLIADCTYRWHVGTWMECSVSCGDGIQRRR | |
| | DTCLGPQAQAPVPADFCQHLPKPVTVRGCWAGPCVGQ | |
| | GTPSLVPHEEAAAPGRTTATPAGASLEWSQARGLLFSP | |
| | APQPRRLLPGPQENSVQSSACGRQHLEPTGTIDMRGPG | |
| | QADCAVAIGRPLGEVVTLRVLESSLNCSAGDMLLLWG | |
| | RLTWRKMCRKLLDMTFSSKTNTLVVRQRCGRPGGGVL | |
| | LRYGSQLAPETFYRECDMQLFGPWGEIVSPSLSPATSNA | |
| | GGCRLFINVAPHARIAIHALATNMGAGTEGANASYILIR | |
| | DTHSLRTTAFHGQQVLYWESESSQAEMEFSEGFLKAQA | |
| | SLRGQYWTLQSWVPEMQDPQSWKGKEGT | |
| SEQ ID NO: | MHQRHPRARCPPLCVAGILACGFLLGCWGPSHFQQSCL | ADAMTS13 |
| 2 | QALEPQAVSSYLSPGAPLKGRPPSPGFQRQRQRQRRAA | (Residues 1- |
| | GGILHLELLVAVGPDVFQAHQEDTERYVLTNLNIGAEL | 745) |
| | LRDPSLGAQFRVHLVKMVILTEPEGAPNITANLTSSLLS | |
| | VCGWSQTINPEDDTDPGHADLVLYITRFDLELPDGNRQ | |
| | VRGVTQLGGACSPTWSCLITEDTGFDLGVTIAHEIGHSF | |
| | GLEHDGAPGSGCGPSGHVMASDGAAPRAGLAWSPCSR | |
| | RQLLSLLSAGRARCVWDPPRPQPGSAGHPPDAQPGLYY | |
| | SANEQCRVAFGPKAVACTFAREHLDMCQALSCHTDPL | |
| | DQSSCSRLLVPLLDGTECGVEKWCSKGRCRSLVELTPIA | |
| | AVHGRWSSWGPRSPCSRSCGGGVVTRRRQCNNPRPAF | |
| | GGRACVGADLQAEMCNTQACEKTQLEFMSQQCARTD | |
| | GQPLRSSPGGASFYHWGAAVPHSQGDALCRHMCRAIG | |
| | ESFIMKRGDSFLDGTRCMPSGPREDGTLSLCVSGSCRTF | |
| | GCDGRMDSQQVWDRCQVCGGDNSTCSPRKGSFTAGR | |
| | AREYVTFLTVTPNLTSVYIANHRPLFTHLAVRIGGRYVV | |
| | AGKMSISPNTTYPSLLEDGRVEYRVALTEDRLPRLEEIRI | |
| | WGPLQEDADIQVYRRYGEEYGNLTRPDITFTYFQPKPR | |
| | QAWVWAAVRGPCSVSCGAGLRWVNYSCLDQARKELV | |
| | ETVQCQGSQQPPAWPEACVLEPCPPY | |
| SEQ ID NO: | MHQRHPRARCPPLCVAGILACGFLLGCWGPSHFQQSCL | ADAMTS 13- |
| 3 | QALEPQAVSSYLSPGAPLKGRPPSPGFQRQRQRQRRAA | GPI anchor |
| | GGILHLELLVAVGPDVFQAHQEDTERYVLTNLNIGAEL | fusion |
| | LRDPSLGAQFRVHLVKMVILTEPEGAPNITANLTSSLLS | protein |
| | VCGWSQTINPEDDTDPGHADLVLYITRFDLELPDGNRQ | |
| | VRGVTQLGGACSPTWSCLITEDTGFDLGVTIAHEIGHSF | |
| | GLEHDGAPGSGCGPSGHVMASDGAAPRAGLAWSPCSR | |
| | RQLLSLLSAGRARCVWDPPRPQPGSAGHPPDAQPGLYY | |
| | SANEQCRVAFGPKAVACTFAREHLDMCQALSCHTDPL | |
| | DQSSCSRLLVPLLDGTECGVEKWCSKGRCRSLVELTPIA | |
| | AVHGRWSSWGPRSPCSRSCGGGVVTRRRQCNNPRPAF | |
| | GGRACVGADLQAEMCNTQACEKTQLEFMSQQCARTD | |
| | GQPLRSSPGGASFYHWGAAVPHSQGDALCRHMCRAIG | |
| | ESFIMKRGDSFLDGTRCMPSGPREDGTLSLCVSGSCRTF | |
| | GCDGRMDSQQVWDRCQVCGGDNSTCSPRKGSFTAGR | |
| | AREYVTFLTVTPNLTSVYIANHRPLFTHLAVRIGGRYVV | |
| | AGKMSISPNTTYPSLLEDGRVEYRVALTEDRLPRLEEIRI | |
| | WGPLQEDADIQVYRRYGEEYGNLTRPDITFTYFQPKPR | |
| | QAWVWAAVRGPCSVSCGAGLRWVNYSCLDQARKELV | |
| | ETVQCQGSQQPPAWPEACVLEPCPPYPNKGSGTTSGTT | |
| | RLLSGHTCFTLTGLLGTLVTMGLLT | |
| SEQ ID NO: | PNKGSGTTSGTTRLLSGHTCFTLTGLLGTLVTMGLLT | GPI anchor |
| 4 | | |
| SEQ ID NO: | atgcaccagcgtcacccccgggcaagatgccctcccctctgtgtggccggaatccttgc | ADAMTS13 |
| 5 | ctgtggattctcctgggctgctggggaccctcccatttccagcagagttgtcttcaggctt | (full length; |
| | tggagccacaggccgtgtatatacttgagccctggtgctcccttaaaaggccgccctc | 1-1427 aa) |
| | cttcccctggcttccagaggcagaggcagaggcagaggcgggctgcaggcggcatc | NM_139025 |
| | ctacacctggagctgctggtggccgtgggccccgatgtatccaggctcaccaggagg | (ADAMTS13 |
| | acacagagcgctatgtgctcaccaacctcaacatcggggcagaactgcttcgggaccc | isoform 1) |
| | gtccctgggggctcagtttcgggtgcacctggtgaagatggtcattctgacagagcctga | |
| | gggtgctccaaatatcacagccaacctcacctcgtccctgctgagcgtctgtgggtgga | |
| | gccagaccatcaaccctgaggacgacacggatcctggccatgctgacctggtcctctat | |
| | atcactaggtttgacctggagttgcctgatggtaaccggcaggtgcggggcgtcaccca | |
| | gctgggcggtgcctgctccccaacctggagctgcctcattaccgaggacactggcttcg | |
| | acctgggagtcaccattgcccatgagattgggcacagatcggcctggagcacgacgg | |
| | cgcgcccggcagcggctgcggccccagcggacacgtgatggcttcggacggcgccg | |
| | cgccccgcgccggcctcgcctggtccccctgcagccgccggcagctgctgagcctgc | |
| | tcagcgcaggacgggcgcgctgcgtgtgggacccgccgcggcctcaacccgggtcc | |
| | gcggggcacccgccggatgcgcagcctggcctctactacagcgccaacgagcagtg | |
| | ccgcgtggccttcggccccaaggctgtcgcctgcaccttcgccagggagcacctggat | |
| | atgtgccaggccctctcctgccacacagacccgctggaccaaagcagctgcagccgc | |
| | ctcctcgttcctctcctggatgggacagaatgtggcgtggagaagtggtgctccaagggt | |
| | cgctgccgctccctggtggagctgacccccatagcagcagtgcatgggcgctggtcta | |
| | gctggggtccccgaagtccttgctcccgctcctgcggaggaggtgtggtcaccaggag | |
| | gcggcagtgcaacaaccccagacctgcctttggggggcgtgcatgtgttggtgctgac | |
| | ctccaggccgagatgtgcaacactcaggcctgcgagaagacccagctggagttcatgt | |
| | cgcaacagtgcgccaggaccgacggccagccgctgcgctcctcccctggcggcgcct | |
| | ccttctaccactggggtgctgctgtaccacacagccaaggggatgctctgtgcagacac | |
| | atgtgccgggccattggcgagagatcatcatgaagcgtggagacagatcctcgatgg | |
| | gacccggtgtatgccaagtggcccccgggaggacgggaccctgagcctgtgtgtgtc | |
| | gggcagctgcaggacatttggctgtgatggtaggatggactcccagcaggtatgggac | |
| | aggtgccaggtgtgtggtggggacaacagcacgtgcagcccacggaagggctattca | |
| | cagctggcagagcgagagaatatgtcacgtttctgacagttacccccaacctgaccagt | |
| | gtctacattgccaaccacaggcctctcttcacacacttggcggtgaggatcggagggcg | |
| | ctatgtcgtggctgggaagatgagcatctcccctaacaccacctacccctccctcctgga | |
| | ggatggtcgtgtcgagtacagagtggccctcaccgaggaccggctgccccgcctgga | |
| | ggagatccgcatctggggacccctccaggaagatgctgacatccaggfttacaggcgg | |
| | tatggcgaggagtatggcaacctcacccgcccagacatcaccttcacctacttccagcct | |
| | aagccacggcaggcctgggtgtgggccgctgtgcgtgggccctgctcggtgagctgt | |
| | ggggcagggctgcgctgggtaaactacagctgcctggaccaggccaggaaggagttg | |
| | gtggagactgtccagtgccaagggagccagcagccaccagcgtggccagaggcctg | |
| | cgtgctcgaaccctgccctccctactgggcggtgggagacttcggcccatgcagcgcc | |
| | tcctgtgggggtggcctgcgggagcggccagtgcgctgcgtggaggcccagggcag | |
| | cctcctgaagacattgcccccagcccggtgcagagcaggggcccagcagccagctgt | |
| | ggcgctggaaacctgcaacccccagccctgccctgccaggtgggaggtgtcagagcc | |
| | cagctcatgcacatcagctggtggagcaggcctggccttggagaacgagacctgtgtg | |
| | ccaggggcagatggcctggaggctccagtgactgaggggcctggctccgtagatgag | |
| | aagctgcctgcccctgagccctgtgtcgggatgtcatgtcctccaggctggggccatct | |
| | ggatgccacctctgcaggggagaaggctccctccccatggggcagcatcaggacggg | |
| | ggctcaagctgcacacgtgtggacccctgcggcagggtcgtgctccgtctcctgcggg | |
| | cgaggtctgatggagctgcgtttcctgtgcatggactctgccctcagggtgcctgtccag | |
| | gaagagctgtgtggcctggcaagcaagcctgggagccggcgggaggtctgccaggc | |
| | tgtcccgtgccctgctcggtggcagtacaagctggcggcctgcagcgtgagctgtggg | |
| | agaggggtcgtgcggaggatcctgtattgtgcccgggcccatggggaggacgatggt | |
| | gaggagatcctgttggacacccagtgccaggggctgcctcgcccggaaccccaggag | |
| | gcctgcagcctggagccctgcccacctaggtggaaagtcatgtcccttggcccatgttc | |
| | ggccagctgtggccttggcactgctagacgctcggtggcctgtgtgcagctcgaccaa | |
| | ggccaggacgtggaggtggacgaggcggcctgtgcggcgctggtgcggcccgagg | |
| | ccagtgtcccctgtctcattgccgactgcacctaccgctggcatgttggcacctggatgg | |
| | agtgctctgtttcctgtggggatggcatccagcgccggcgtgacacctgcctcggaccc | |
| | caggcccaggcgcctgtgccagctgatttctgccagcacttgcccaagccggtgactgt | |
| | gcgtggctgctgggctgggccctgtgtgggacagggtacgcccagcctggtgcccca | |
| | cgaagaagccgctgctccaggacggaccacagccacccctgctggtgcctccctgga | |
| | gtggtcccaggcccggggcctgctcttctccccggctccccagcctcggcggctcctg | |
| | cccgggccccaggaaaactcagtgcagtccagtgcctgtggcaggcagcaccttgag | |
| | ccaacaggaaccattgacatgcgaggcccagggcaggcagactgtgcagtggccatt | |
| | gggcggcccctcggggaggtggtgaccctccgcgtccttgagagttctctcaactgca | |
| | gtgcgggggacatgttgctgattggggccggctcacctggaggaagatgtgcaggaa | |
| | gctgttggacatgactttcagctccaagaccaacacgctggtggtgaggcagcgctgcg | |
| | ggcggccaggaggtggggtgctgctgcggtatgggagccagcttgctcctgaaacctt | |
| | ctacagagaatgtgacatgcagctattgggccctggggtgaaatcgtgagcccctcgct | |
| | gagtccagccacgagtaatgcagggggctgccggctcttcattaatgtggctccgcacg | |
| | cacggattgccatccatgccctggccaccaacatgggcgctgggaccgagggagcca | |
| | atgccagctacatcttgatccgggacacccacagcttgaggaccacagcgttccatggg | |
| | cagcaggtgctctactgggagtcagagagcagccaggctgagatggagttcagcgag | |
| | ggcttcctgaaggctcaggccagcctgcggggccagtactggaccctccaatcatggg | |
| | taccggagatgcaggaccctcagtcctggaagggaaaggaaggaacctga | |
| SEQ ID NO: | atgcaccagcgtcacccccgggcaagatgccctcccctctgtgtggccggaatccttgc | ADAMTS13 |
| 6 | ctgtggattctcctgggctgctggggaccctcccatttccagcagagttgtcttcaggctt | (Residues 1- |
| | tggagccacaggccgtgtcttcttacttgagccctggtgctcccttaaaaggccgccctc | 745) |
| | cttcccctggcttccagaggcagaggcagaggcagaggcgggctgcaggcggcatc | |
| | ctacacctggagctgctggtggccgtgggccccgatgtatccaggctcaccaggagg | |
| | acacagagcgctatgtgctcaccaacctcaacatcggggcagaactgcttcgggaccc | |
| | gtccctgggggctcagtttcgggtgcacctggtgaagatggtcattctgacagagcctga | |
| | gggtgctccaaatatcacagccaacctcacctcgtccctgctgagcgtctgtgggtgga | |
| | gccagaccatcaaccctgaggacgacacggatcctggccatgctgacctggtcctctat | |
| | atcactaggtttgacctggagttgcctgatggtaaccggcaggtgcggggcgtcaccca | |
| | gctgggcggtgcctgctccccaacctggagctgcctcattaccgaggacactggcttcg | |
| | acctgggagtcaccattgcccatgagattgggcacagcttcggcctggagcacgacgg | |
| | cgcgcccggcagcggctgcggccccagcggacacgtgatggcttcggacggcgccg | |
| | cgccccgcgccggcctcgcctggtccccctgcagccgccggcagctgctgagcctgc | |
| | tcagcgcaggacgggcgcgctgcgtgtgggacccgccgcggcctcaacccgggtcc | |
| | gcggggcacccgccggatgcgcagcctggcctctactacagcgccaacgagcagtg | |
| | ccgcgtggccttcggccccaaggctgtcgcctgcaccttcgccagggagcacctggat | |
| | atgtgccaggccctctcctgccacacagacccgctggaccaaagcagctgcagccgc | |
| | ctcctcgttcctctcctggatgggacagaatgtggcgtggagaagtggtgctccaagggt | |
| | cgctgccgctccctggtggagctgacccccatagcagcagtgcatgggcgctggtcta | |
| | gctggggtccccgaagtccttgctcccgctcctgcggaggaggtgtggtcaccaggag | |
| | gcggcagtgcaacaaccccagacctgcctttggggggcgtgcatgtgttggtgctgac | |
| | ctccaggccgagatgtgcaacactcaggcctgcgagaagacccagctggagttcatgt | |
| | cgcaacagtgcgccaggaccgacggccagccgctgcgctcctcccctggcggcgcct | |
| | ccttctaccactggggtgctgctgtaccacacagccaaggggatgctctgtgcagacac | |
| | atgtgccgggccattggcgagagatcatcatgaagcgtggagacagatcctcgatgg | |
| | gacccggtgtatgccaagtggcccccgggaggacgggaccctgagcctgtgtgtgtc | |
| | gggcagctgcaggacatttggctgtgatggtaggatggactcccagcaggtatgggac | |
| | aggtgccaggtgtgtggtggggacaacagcacgtgcagcccacggaagggctattca | |
| | cagctggcagagcgagagaatatgtcacgtttctgacagttacccccaacctgaccagt | |
| | gtctacattgccaaccacaggcctctcttcacacacttggcggtgaggatcggagggcg | |
| | ctatgtcgtggctgggaagatgagcatctcccctaacaccacctacccctccctcctgga | |
| | ggatggtcgtgtcgagtacagagtggccctcaccgaggaccggctgccccgcctgga | |
| | ggagatccgcatctggggacccctccaggaagatgctgacatccaggfttacaggcgg | |
| | tatggcgaggagtatggcaacctcacccgcccagacatcaccttcacctacttccagcct | |
| | aagccacggcaggcctgggtgtgggccgctgtgcgtgggccctgctcggtgagctgt | |
| | ggggcagggctgcgctgggtaaactacagctgcctggaccaggccaggaaggagttg | |
| | gtggagactgtccagtgccaagggagccagcagccaccagcgtggccagaggcctg | |
| | cgtgctcgaaccctgccctccctac | |
| SEQ ID NO: | atgcaccagcgtcacccccgggcaagatgccctcccctctgtgtggccggaatccttgc | ADAMTS13- |
| 7 | ctgtggattctcctgggctgctggggaccctcccatttccagcagagttgtcttcaggctt | GPI anchor |
| | tggagccacaggccgtgtatatacttgagccctggtgctcccttaaaaggccgccctc | fusion |
| | cttcccctggcttccagaggcagaggcagaggcagaggcgggctgcaggcggcatc | protein |
| | ctacacctggagctgctggtggccgtgggccccgatgtatccaggctcaccaggagg | |
| | acacagagcgctatgtgctcaccaacctcaacatcggggcagaactgcttcgggaccc | |
| | gtccctgggggctcagtttcgggtgcacctggtgaagatggtcattctgacagagcctga | |
| | gggtgctccaaatatcacagccaacctcacctcgtccctgctgagcgtctgtgggtgga | |
| | gccagaccatcaaccctgaggacgacacggatcctggccatgctgacctggtcctctat | |
| | atcactaggtttgacctggagttgcctgatggtaaccggcaggtgcggggcgtcaccca | |
| | gctgggcggtgcctgctccccaacctggagctgcctcattaccgaggacactggcttcg | |
| | acctgggagtcaccattgcccatgagattgggcacagatcggcctggagcacgacgg | |
| | cgcgcccggcagcggctgcggccccagcggacacgtgatggcttcggacggcgccg | |
| | cgccccgcgccggcctcgcctggtccccctgcagccgccggcagctgctgagcctgc | |
| | tcagcgcaggacgggcgcgctgcgtgtgggacccgccgcggcctcaacccgggtcc | |
| | gcggggcacccgccggatgcgcagcctggcctctactacagcgccaacgagcagtg | |
| | ccgcgtggccttcggccccaaggctgtcgcctgcaccttcgccagggagcacctggat | |
| | atgtgccaggccctctcctgccacacagacccgctggaccaaagcagctgcagccgc | |
| | ctcctcgttcctctcctggatgggacagaatgtggcgtggagaagtggtgctccaagggt | |
| | cgctgccgctccctggtggagctgacccccatagcagcagtgcatgggcgctggtcta | |
| | gctggggtccccgaagtccttgctcccgctcctgcggaggaggtgtggtcaccaggag | |
| | gcggcagtgcaacaaccccagacctgcctttggggggcgtgcatgtgttggtgctgac | |
| | ctccaggccgagatgtgcaacactcaggcctgcgagaagacccagctggagttcatgt | |
| | cgcaacagtgcgccaggaccgacggccagccgctgcgctcctcccctggcggcgcct | |
| | ccttctaccactggggtgctgctgtaccacacagccaaggggatgctctgtgcagacac | |
| | atgtgccgggccattggcgagagatcatcatgaagcgtggagacagatcctcgatgg | |
| | gacccggtgtatgccaagtggcccccgggaggacgggaccctgagcctgtgtgtgtc | |
| | gggcagctgcaggacatttggctgtgatggtaggatggactcccagcaggtatgggac | |
| | aggtgccaggtgtgtggtggggacaacagcacgtgcagcccacggaagggctattca | |
| | cagctggcagagcgagagaatatgtcacgtttctgacagttacccccaacctgaccagt | |
| | gtctacattgccaaccacaggcctctatcacacacttggcggtgaggatcggagggcg | |
| | ctatgtcgtggctgggaagatgagcatctcccctaacaccacctacccctccctcctgga | |
| | ggatggtcgtgtcgagtacagagtggccctcaccgaggaccggctgccccgcctgga | |
| | ggagatccgcatctggggacccctccaggaagatgctgacatccaggtttacaggcgg | |
| | tatggcgaggagtatggcaacctcacccgcccagacatcaccttcacctacttccagcct | |
| | aagccacggcaggcctgggtgtgggccgctgtgcgtgggccctgctcggtgagctgt | |
| | ggggcagggctgcgctgggtaaactacagctgcctggaccaggccaggaaggagttg | |
| | gtggagactgtccagtgccaagggagccagcagccaccagcgtggccagaggcctg | |
| | cgtgctcgaaccctgccctccctacCCAAATAAAGGAAGTGGAAC | |
| | CACTTCAGGTACTACCCGTCTTCTATCTGGGCACA | |
| | CGTGTTTCACGTTGACAGGTTTGCTTGGGACGCTA | |
| | GTAACCATGGGCTTGCTGACT | |
| SEQ ID NO: | CCAAATAAAGGAAGTGGAACCACTTCAGGTACTACC | GPI anchor |
| 8 | CGTCTTCTATCTGGGCACACGTGTTTCACGTTGACAG | |
| | GTTTGCTTGGGACGCTAGTAACCATGGGCTTGCTGAC | |
| | T | |

### (a) ADAMVTS13 and Variants Thereof

An “ADAMVTS13 protein,” as used herein, refers to any protein or polypeptide with ADAMVTS13 activity, particularly the ability to cleave the peptide bond between residues Tyr-842 and Met-843 of VWF. For example, an ADAMTS13 protein may be a polypeptide comprising an amino acid sequence having significant identity to that of NP_620594 (ADAMTS13 isoform 1, preproprotein; SEQ TD NO: 1) or amino acids 75 to 1427 of NP_620594 (ADAMTS13 isoform 1, mature polypeptide; SEQ TD NO: 9). In another example, an ADAMVTS13 protein refers to a polypeptide comprising an amino acid sequence having significant identity to that of NP_620596 (ADAMTS13 isoform 2, preproprotein; SEQ ID NO: 10) or amino acids 75 to 1371 of NP_620596 (ADAMTS13 isoform 2, mature polypeptide; SEQ ID NO: 11). ADAMTS13 proteins may also include polypeptides comprising an amino acid sequence having significant identity to that of NP_620595 (ADAMVTS13 isoform 3, preproprotein; SEQ TD NO: 12) or amino acids 75 to 1340 of NP_620595 (ADAMTS13 isoform 1, mature polypeptide; SEQ TD NO: 13). As used herein, an ADAMVTS13 protein includes natural variants with VWF cleaving activity and artificial constructs with VWF cleaving activity. As used herein, ADAMTS13 encompasses any natural variants, alternative sequences, isoforms or mutant proteins that retain some basal activity. Examples of ADAMTS13 mutations found in the human population include, without limitation, R7W, V88M, H96D, R102C, R193W, T1961, H234Q, A250V, R268P, W390C, R398H, Q448E, Q456H, P457L, C508Y, R528G, P618A, R625H, 1673F, R692C, A732V, S903L, C908Y, C951G, G982R, C1024G, A1033T, R1095W, R1123C, C1213Y, T12261, G1239V, R1336W, many of which have been found associated with TTP. ADAMTS13 proteins also include polypeptides containing post-translational modifications. For example, ADAMTS13 has been shown to be modified by N-acetylglucosamine (GlcNAc) at residues 614, 667, and 1354, and it has been predicted that residues 142, 146, 552, 579, 707, 828, and 1235 may also be modified in this fashion.

In particular, the term “ADAMTS13 gene” refers to a full-length ADAMTS13 nucleotide sequence (e.g., as shown in SEQ ID NO: 5). However, the term also encompasses fragments of the ADAMTS13 sequence, as well as other domains with the full-length ADAMTS13 nucleotide sequence. Furthermore, the term “ADAMTS13 nucleotide sequence” or “ADAMTS13 polynucleotide sequence” encompasses DNA, cDNA, and RNA (e.g., mRNA) sequences.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

In some embodiments, ADAMTS13 variants may contain one or more mutations in a region recognized by auto-antibodies. The one or more mutations may reduce or abolish the interactions between ADAMTS13 variants and auto-antibodies. Such ADAMTS13 variants may be resistant to the inhibitors (e.g., auto-antibodies), which are responsible for the acquired form of TTP. The regions recognized by auto-antibodies may include known epitopes revealed by epitope mapping in the TTP patients. Such regions can be located in the catalytic domain or other parts of ADAMTS13. In some embodiments, ADAMTS13 variants remain enzymatically active against VWF and are not inhibited by common auto-antibodies.

### (b) Lipid Anchor

In some embodiments, the fusion protein is a lipid-anchored protein. For example, the fusion protein may include a lipid anchor operably linked to the fragment of ADAMTS13 or its variants. The lipid anchor may be operably linked to the C-terminal end of the fragment of ADAMTS13. The lipid anchor can be a Glycosylphosphatidylinositol (GPI) anchor. The GPI anchor may include the amino acid sequence of SEQ ID NO: 4.

Lipid-anchored proteins (also known as lipid-linked proteins) are proteins located on the surface of the cell membrane that are covalently attached to lipids embedded within the cell membrane. These proteins insert and assume a place in the bilayer structure of the membrane alongside the similar fatty acid tails. The lipid-anchored protein can be located on either side of the cell membrane. Thus, the lipid serves to anchor the protein to the cell membrane. The lipid groups play a role in protein interaction and can contribute to the function of the protein to which it is attached. Furthermore, the lipid serves as a mediator of membrane associations or as a determinant for specific protein-protein interactions. For example, lipid groups can play an important role in increasing molecular hydrophobicity. This allows for the interaction of proteins with cellular membranes and protein domains.

Lipid-anchored proteins may include prenylated proteins, fatty acylated proteins, and glycosylphosphatidylinositol (GPI)-linked proteins. A protein can have multiple lipid groups covalently attached to it, but the site where the lipid binds to the protein depends both on the lipid group and protein.

Glycosylphosphatidylinositols (GPI) proteins are attached to a GPI complex molecular group via an amide linkage to the protein's C-terminal carboxyl group. This GPI complex consists of several main components that are all interconnected: a phosphoethanolamine, a linear tetrasaccharide (composed of three mannose and a glucosaminyl) and a phosphatidylinositol. The phosphatidylinositol group is glycosidically linked to the non-N-acetylated glucosamine of the tetrasaccharide. A phosphodiester bond is then formed between the mannose at the nonreducing end (of the tetrasaccharide) and the phosphoethanolamine. The phosphoethanolamine is then amide linked to the C-terminal of the carboxyl group of the protein. The GPI attachment occurs through the action of GPI-transamidase complex. The fatty acid chains of the phosphatidylinositol are inserted into the membrane and thus are what anchor the protein to the membrane. These proteins are only located on the exterior surface of the plasma membrane.

### (c) Preparation of Genetically Modified Red Blood Cells

In some embodiments, this disclosure also provides a method for preparing the above-described red blood cells. The method includes: (i) providing a plurality of stem cells; (ii) contacting the stem cells with a nucleic acid encoding a fusion protein comprising ADAMTS13 or fragment thereof to obtain transduced stem cells; (iii) expanding the transduced stem cells cells in cell culture medium; and (iv) collecting the resulting red blood cells.

The term “expanding” or “culturing” refers to maintaining or cultivating cells under conditions in which they can proliferate and avoid senescence. For example, cells may be cultured in media optionally containing one or more growth factors, i.e., a growth factor cocktail. Stable cell lines may be established to allow for continued propagation of cells.

In some embodiments, red blood cells are cultured/expanded in a hollow fiber bioreactor to produce red blood cells at a large scale, for example, at a density of 5×10⁸ cell/mL, which is sufficient to perform a small clinical trial.

In some embodiments, the red blood cell is transduced with a retrovirus comprising a nucleic acid encoding the fusion protein. The nucleic acid may include a nucleotide sequence at least 75% identical to a nucleic acid sequence of SEQ ID NOs: 5, 6, or 7.

The expression of the fusion protein can be induced by introducing one or more expression vectors carrying nucleic acids encoding an ADAMTS13 polypeptide or fragment thereof. The ADAMTS13 polypeptide or fragment thereof can be inserted into the proper site of the vector (e.g., operably linked to a promoter). The expression vector is introduced into a selected host cell (e.g., red blood cell) for amplification and/or polypeptide expression, by well-known methods such as transfection, transduction, infection, electroporation, microinjection, lipofection or the DEAE-dextran method or other known techniques. These methods and other suitable methods are well known to the skilled artisan.

A wide variety of vectors can be used for the expression of the fusion protein as described. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., red blood cells). Accordingly, in certain embodiments, a viral vector is used to introduce a nucleotide sequence encoding an ADAMTS13 protein or fragment thereof into a host cell for expression. The viral vector will comprise a nucleotide sequence encoding an ADAMTS13 protein or fragment thereof operably linked to one or more control sequences, for example, a promoter. Alternatively, the viral vector may not contain a control sequence and will instead rely on a control sequence within the host cell to drive expression of the ADAMTS13 protein or fragment thereof. Non-limiting examples of viral vectors that may be used to deliver a nucleic acid include adenoviral vectors, AAV vectors, and retroviral vectors.

In some embodiments, an adeno-associated virus (AAV) can be used to introduce a nucleotide sequence encoding an ADAMTS13 protein or fragment thereof into a host cell for expression. AAV systems have been described previously and are generally well known in the art (Kelleher and Vos, Biotechniques, 17(6):1110-7, 1994; Cotten et al., Proc Natl Acad Sci USA, 89(13):6094-6098, 1992; Curiel, Nat Immun, 13(2-3):141-64, 1994; Muzyczka, Curr Top Microbiol Immunol, 158:97-129, 1992). Details concerning the generation and use of rAAV vectors are described, for example, in U.S. Pat. Nos. 5,139,941 and 4,797,368, each incorporated herein by reference in its entirety for all purposes.

In some embodiments, a retroviral expression vector can be used to introduce a nucleotide sequence encoding an ADAMTS13 protein or fragment thereof into a host cell for expression. These systems have been described previously and are generally well known in the art (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986).

Examples of vectors for eukaryotic expression in mammalian cells include AD5, pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g., 24-73 hours, or for at least two weeks, and may be allowed to grow for five weeks or more, before analysis. Commonly used retroviral vectors are “defective,” i.e., unable to produce viral proteins required for productive infection. Replication of the vector requires growth in the packaging cell line. The host cell specificity of the retrovirus is determined by the envelope protein, env (pl20). The envelope protein is provided by the packaging cell line. Envelope proteins are of at least three types, ecotropic, amphotropic and xenotropic. Retroviruses packaged with ecotropic envelope protein, e.g., MMLV, are capable of infecting most murine and rat cell types. Ecotropic packaging cell lines include BOSC23. Retroviruses bearing amphotropic envelope protein, e.g., 4070A, are capable of infecting most mammalian cell types, including human, dog, and mouse. Amphotropic packaging cell lines include PA12 and PA317. Retroviruses packaged with xenotropic envelope protein, e.g., AKR env, are capable of infecting most mammalian cell types, except murine cells. The vectors may include genes that must later be removed, e.g., using a recombinase system such as Cre/Lox, or the cells that express them destroyed, e.g., by including genes that allow selective toxicity such as herpesvirus TK, bcl-xs, etc. Suitable inducible promoters are activated in a desired target cell type, either the transfected cell or progeny thereof.

In some embodiments, genome-editing techniques, such as CRISPR/Cas9 systems, designer zinc fingers, transcription activator-like effectors (TALEs), or homing meganucleases are available to induce expression of the describe fusion protein in a red blood cell. In general, “CRISPR/Cas9 system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more elements of a CRISPR system may be derived from a type I, type II, or type III CRISPR system. Alternatively, one or more elements of a CRISPR system may be derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

Mature red blood cells for use in generating the modified red blood cells may be isolated using various methods such as, for example, a cell washer, a continuous flow cell separator, density gradient separation, fluorescence-activated cell sorting (FACS), Miltenyi immunomagnetic depletion (MACS), or a combination of these methods.

Alternatively, red blood cells may be isolated using density gradient centrifugation with various separation mediums such as, for example, Ficoll, Hypaque, Histopaque, Percoll, Sigmacell, or combinations thereof. Red blood cells may also be isolated by centrifugation using a Percoll step gradient. As such, fresh blood is mixed with an anticoagulant solution and the cells washed briefly in Hepes-buffered saline. Leukocytes and platelets are removed by adsorption with a mixture of α-cellulose and Sigmacell. The red blood cells are further isolated from reticulocytes and residual white blood cells by centrifugation through a Percoll step gradient. The red blood cells are recovered in the pellet while reticulocytes and the remaining white blood cells band at different interfaces.

Red blood cells may be separated from reticulocytes, for example, using flow cytometry. In this instance, whole blood is centrifuged to separate cells from plasma. The cell pellet is resuspended in phosphate buffered saline solution and further fractionated on Ficoll-Paque, for example, by centrifugation to separate the red blood cells from the white blood cells. The resulting cell pellet is resuspended in RPMI supplemented with 10% fetal bovine serum and sorted on a FACS instrument based on size and granularity.

Red blood cells may be isolated by immunomagnetic depletion. In this instance, magnetic beads with cell-type specific antibodies are used to eliminate non-red blood cells. For example, red blood cells are isolated from the majority of other blood components using a density gradient as described above followed by immunomagnetic depletion of any residual reticulocytes. The cells are pre-treated with human antibody serum and then treated antibodies against reticulocyte specific antigens such as, for example, CD71 and CD36. The antibodies may be directly attached to magnetic beads or conjugated to PE, for example, to which magnetic beads with anti-PE antibody will react. As such, the antibody-magnetic bead complex can selectively extract residual reticulocytes, for example, from the red blood cell population.

Red blood cells may also be isolated using apheresis. The process of apheresis involves removal of whole blood from a patient or donor, separation of blood components using centrifugation or cell sorting, withdrawal of one or more of the separated portions, and transfusion of remaining components back into the patient or donor. A number of instruments are currently in use for this purpose such as, for example, the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods, such as those described above, may be necessary to achieve the appropriate degree of red blood cell purity.

The modified red blood cells can be autologous and/or allogeneic to the subject. In some embodiments, erythrocytes allogeneic to the subject include one or more of one or more blood type specific erythrocytes or one or more universal donor erythrocytes. The modified red blood cells can be fusion erythrocytes between erythrocytes autologous to the subject and one or more allogeneic erythrocytes, liposomes, and/or artificial vesicles.

For autologous transfusion, red blood cells from an individual are isolated and modified by methods described herein and retransfused into the individual. For allogeneic transfusions, red blood cells are isolated from a donor, modified by methods described herein and transfused into another individual.

### (d) Additional Agents

In some embodiments, red blood cells may additionally be loaded with one or more molecular agents. Such molecular agents can be internalized within the red blood cell and may include, but is not limited to, a compound that is configured to provide an activity to the subject and/or to the red blood cell following administration. In some embodiments, such molecular agents may include, but are not limited to, one or more therapeutic agents or imaging agents.

A number of methods may be used to load modified red blood cells with a molecular agent, such as hypotonic lysis, hypotonic dialysis, osmosis, osmotic pulsing, osmotic shock, ionophoresis, electroporation, sonication, microinjection, calcium precipitation, membrane intercalation, lipid-mediated transfection, detergent treatment, viral infection, diffusion, receptor-mediated endocytosis, use of protein transduction domains, particle firing, membrane fusion, freeze-thawing, mechanical disruption, and filtration.

In some embodiments, the molecular agent can be a therapeutic agent, such as a small molecule drug or biological effector molecule. Therapeutic agents of interest include, without limitation, pharmacologically active drugs, genetically active molecules, etc. Therapeutic agents of interest include antineoplastic agents, anti-inflammatory agents, hormones or hormone antagonists, ion channel modifiers, and neuroactive agents.

Small molecules, including inorganic and organic chemicals, may also be used. In an embodiment, the small molecule is a pharmaceutically active agent. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumor suppressors).

If a prodrug is loaded in an inactive form, a second effector molecule may be loaded into a modified red blood cell or a red blood cell that is to be modified according to the disclosure herein. Such a second effector molecule is usefully an activating polypeptide which converts the inactive prodrug to active drug form. In an embodiment, activating polypeptides include, but are not limited to, viral thymidine kinase, carboxypeptidase A, α-galactosidase, β-glucuronidase, alkaline phosphatase, or cytochrome P-450, plasmin, carboxypeptidase G2, cytosine deaminase, glucose oxidase, xanthine oxidase, β-glucosidase, azoreductase, t-glutamyl transferase, β-lactamase, or penicillin amidase.

Either the polypeptide or the gene encoding it may be loaded into the modified, or to-be-modified, red blood cells; if the latter, both the prodrug and the activating polypeptide may be encoded by genes on the same recombinant nucleic acid construct. Furthermore, either the prodrug or the activator of the prodrug may be already loaded into the red blood cell. The relevant activator or prodrug (as the case may be) is then loaded as a second agent according to the methods described herein.

The therapeutic agent may also be a biological effector molecule which has activity in a biological system. Biological effector molecules, include, but are not limited to, a protein, polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanized, a peptide hormone, a receptor, or a signaling molecule. Included within the term “immunoglobulin” are intact immunoglobulins as well as antibody fragments such as Fv, a single chain Fv (scFv), a Fab or a F (ab′)₂.

The biological effector molecules can be immunoglobulins, antibodies, Fv fragments, etc., that are capable of binding to antigens in an intracellular environment. These types of molecules are known as “intrabodies” or “intracellular antibodies.” An “intracellular antibody” or an “intrabody” includes an antibody that is capable of binding to its target or cognate antigen within the environment of a cell, or in an environment that mimics an environment within the cell. Selection methods for directly identifying such “intrabodies” include the use of an in vivo two-hybrid system for selecting antibodies with the ability to bind to antigens inside mammalian cells. Such methods are described in PCT/GB00/00876, incorporated herein by reference.

The biological effector molecule includes, but is not limited to, at least one of a protein, a polypeptide, a peptide, a nucleic acid, a virus, a virus-like an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically). A biological effector molecule may include a nucleic acid, including, but not limited to, an oligonucleotide or modified oligonucleotide, an antisense oligonucleotide or modified antisense oligonucleotide, an aptamer, a cDNA, genomic DNA, an artificial or natural chromosome (e.g., a yeast artificial chromosome) or a part thereof, RNA, including an siRNA, a shRNA, mRNA, tRNA, rRNA or a ribozyme, or a peptide nucleic acid (PNA); a virus or virus-like particles; a nucleotide or ribonucleotide or synthetic analogue thereof, which may be modified or unmodified.

The biological effector molecule can also be an amino acid or analog thereof, which may be modified or unmodified or a non-peptide (e.g., steroid) hormone; a proteoglycan; a lipid; or a carbohydrate. If the biological effector molecule is a polypeptide, it can be loaded directly into a modified red blood cell, according to the methods described herein. Alternatively, a nucleic acid molecule bearing a sequence encoding a polypeptide, which sequence is operatively linked to transcriptional and translational regulatory elements active in a cell at a target site, may be loaded.

The molecular agent may be an imaging agent, which may be detected, whether in vitro or in vivo in the context of a tissue, organ or organism. Examples of agents include those useful for imaging of tissues in vivo or ex vivo. For example, imaging agents, such as labeled antibodies which are specific for defined molecules, tissues or cells in an organism, may be used to image specific parts of the body by releasing from the loaded red blood cells at a desired location using electromagnetic radiation. In some embodiments, the imaging agent emits a detectable signal, such as visible light or other electromagnetic radiation. The imaging agent can be a radioisotope, e.g., ³²P or ³⁵S or ⁹⁹Tc, or a quantum dot, or a molecule such as a nucleic acid, polypeptide, or other molecules, conjugated with such a radioisotope. The imaging agent can be opaque to radiation, such as X-ray radiation. In another embodiment, the imaging agent comprises a targeting functionality by which it is directed to a particular cell, tissue, organ or other compartments within the body of an animal. For example, the agent may comprise a radiolabelled antibody which specifically binds to defined molecule(s), tissue(s) or cell(s) in an organism.

The modified red blood cells may also be labeled with one or more positive markers that can be used to monitor over time the number or concentration of modified red blood cells in the blood circulation of an individual. It is anticipated that the overall number of modified red blood cells will decay over time following initial transfusion. As such, it may be appropriate to correlate the signal from one or more positive markers with that of the activated molecular marker, generating a proportionality of signal that will be independent of the number of modified red blood cells remaining in the circulation. There are presently several fluorescent compounds, for example, that are approved by the Food & Drug Administration for human use including but not limited to fluorescein, indocyanine green, and rhodamine B. For example, red blood cells may be non-specifically labeled with fluorescein isothiocyanate.

### II. USES OF GENETICALLY MODIFIED RED BLOOD CELLS

### (i) Pharmaceutical Compositions

The modified red blood cells can be incorporated into pharmaceutical compositions suitable for administration. The pharmaceutical compositions generally comprise substantially purified modified red blood cells and a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the modified red blood cells, use thereof in the compositions is contemplated. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating the modified red blood cells in the required amount in an appropriate solvent with one or a combination of ingredients enumerated above, as required. Generally, dispersions are prepared by incorporating the modified red blood cells into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The modified red blood cells can be administered in the form of a depot injection or implant preparation which can be formulated in such a manner as to permit a sustained or pulsatile release of the active ingredient.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. For transdermal administration, the modified red blood cells are formulated into ointments, salves, gels, or creams as generally known in the art.

In some embodiments, the modified red blood cells are prepared with carriers that will protect the modified red blood cells against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers.

In some embodiments, the composition includes the red blood cells as described above and optionally a cryo-protectant (e.g., glycerol, DMSO, PEG).

Also within the scope of this disclosure is a kit comprising the modified red blood cells or the composition described above. The kit may further include instructions for administrating the modified red blood cells or the composition and optionally an adjuvant.

In another aspect, this disclosure also provides blood, cellular and acellular blood components, or blood products obtained from the red blood cell as described above.

### (ii) Methods of Treatment

In another aspect, this disclosure also provides a method for treating TTP. In another aspect, a method for increasing a level of functional ADAMTS13 in a subject as well as a method for decreasing aggregation of VWR in a subject are provided. These methods include administering an effective amount of the disclosed red blood cells to the subject. The method includes administering a therapeutically effective amount of the genetically modified red blood cells to a subject in need thereof. TTP may include hereditary TTP, congenital TTP, acquired TTP, or immune-mediated TTP.

The term “thrombotic thrombocytopenic purpura” or “TTP” refers to a disease characterized by intravascular destruction of erythrocytes and consumption of blood platelets resulting in anemia and thrombocytopenia. Diffuse platelet rich microthrombi are observed in multiple organs, with the major extravascular manifestations including fever, and variable degrees of neurologic and renal dysfunction. Purpura refers to the characteristic bleeding that occurs beneath the skin, or in mucous membranes, which produces bruises, or a red rash-like appearance.

The red blood cells can be administered by infusion. In some embodiments, the method may include producing the red blood cells in vitro before administrating to the subject. In some embodiments, the red blood cells can be produced in a hollow fiber culturing system by expansion of hematopoietic progenitors.

The red blood cells may be administered in a pharmaceutical formulation as described above. The dose of the modified red blood cells for an optimal therapeutic benefit can be determined clinically. A certain length of time is allowed to pass for the circulating or locally delivered modified red blood cells. The waiting period will be determined clinically and may vary depending on the composition of the composition.

The cells can be administered to individuals through infusion or injection (for example, intravenous, intrathecal, intramuscular, intraluminal, intratracheal, intraperitoneal, or subcutaneous), transdermally, or other methods known in the art. Administration may be once every two weeks, once a week, or more often, but the frequency may be decreased during a maintenance phase of the disease or disorder.

Both heterologous and autologous cells can be used. In the former case, HLA-matching should be conducted to avoid or minimize host reactions. In the latter case, autologous cells are enriched and purified from a subject and stored for later use. The cells may be cultured in the presence of host or graft T cells ex vivo and re-introduced into the host. This may have the advantage of the host recognizing the cells as self and better providing reduction in T cell activity. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on the recession of pathological signs and clinical and subclinical symptoms of a disease condition or disorder contemplated for treatment with the above-described composition. Dosages and administration regimens can be adjusted depending on the age, sex, physical condition of administered as well as the benefit of the treatment and side effects in the patient or mammalian subject to be treated and the judgment of the physician, as is appreciated by those skilled in the art. In all of the above-described methods, the cells can be administered to a subject at 1×10⁴ to 1×10¹⁰/time.

As used herein, the term “subject” refers to a vertebrate, and in some exemplary aspects, a mammal. Such mammals include, but are not limited to, mammals of the order Rodentia, such as mice and rats, and mammals of the order Lagomorpha, such as rabbits, mammals from the order Carnivora, including Felines (cats) and canines (dogs), mammals from the order Artiodactyla, including bovines (cows) and swines (pigs) or of the order Perissodactyla, including Equines (horses), mammals from the order Primates, Ceboids, or Simoids (monkeys) and of the order Anthropoids (humans and apes). In exemplary aspects, the mammal is a mouse. In more exemplary aspects, the mammal is a human.

As used herein, the term “effective amount” or “therapeutically effective amount” refers to an amount which results in measurable amelioration of at least one symptom or parameter of a specific disorder. A therapeutically effective amount of the above-described cells can be determined by methods known in the art. An effective amount for treating a disorder can be determined by empirical methods known to those of ordinary skill in the art. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of the above-described disorders is within the scope of the invention. Clinically significant attenuation or amelioration means perceptible to the patient and/or to the physician.

### III. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the terms “fragment of ADAMTS13” or “portion of ADAMTS13” refer to an amino acid sequence comprising at least about 5%, 10%, 20%, 30%, 40%, 50%, 60%, 70%, 80%, 90%, 95%, 96%, 97%, 98%, 99% or 100% of a naturally occurring ADAMTS13 proteins or variants/mutants thereof.

The term “fusion” in reference to ADAMTS13 fusion proteins includes, but is not limited to, attachment of at least one lipid anchor, therapeutic protein, polypeptide or peptide to the N-terminal end or the C-terminal end of ADAMTS13, and/or insertion between any two amino acids within ADAMTS13.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “polypeptide,” “peptide,” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, by disulfide bond formation, glycosylation, lipidation, acetylation, phosphorylation, or any other manipulation, such as conjugation with a labeling component. As used herein the term “amino” acid” includes natural and/or unnatural or synthetic amino acids, including glycine and both the D or L optical isomers, and amino acid analogs and peptidomimetics.

The term “amino acid sequence” refers to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns, therefore, are absent in the messenger RNA (mRNA) transcript. The mRNA functions during translation to specify the sequence or order of amino acids in a nascent polypeptide.

The term “recombinant” when made in reference to a nucleic acid molecule refers to a nucleic acid molecule which is comprised of segments of nucleic acid joined together by means of molecular biological techniques. The term “recombinant,” when made in reference to a protein or a polypeptide, refers to a protein molecule which is expressed using a recombinant nucleic acid molecule.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### IV. EXAMPLES

### Example 1

This example describes the materials and methods used in the following examples.

### ADAMTS13-GPI Construct

The cDNA coding first 745 amino acids of the ADAMTS13 protein joined with the DAF gene GPI anchor sequence (37 amino acids) were synthesized by GeneScript (Piscataway, N.J.). The synthesized fusion gene sequence then cloned into a derivative of the pZDonor-AAVS1 Puro vector (Sigma-Aldrich, The Woodlands, Tex.) containing the mini-LCR, alpha-globin promoter and alpha-globin gene by replacing alpha-globin gene region via AatII and SgrdI restriction sites. The plasmid contains homologous arms to the AAVS1 safe harbor site.

### CRISPR-Cas9 RNP

The crRNA targeting the human AAVS1 safe harbor site designed using an online selection tool CRISPOR (Haeussler et al. (2016); Genome Biol. 17, 148) and synthesized (sequence: G*U*C*CCUAGUGGCCCCACUGU) with attached modified EZ linker (Synthego, Redwood City, Calif.). The nucleotides marked with asterisks have 2′-O-methyl analogs and 3′-phosphorothioate internucleotide linkages.

To make the gRNA the crRNA was annealed with the universal tracrRNA (Synthego, Redwood City, Calif.) in a 2:1 ratio. Annealing reaction included incubation of crRNA and tracrRNA at 78° C. for 10 minutes and then at 37° C. for 30 minutes. The reaction was cooled down to room temperature by ramping for 30 minutes on a thermocycler. The final concentration of the annealed gRNA was 30 μM (30 pmol/μl). 150 pmol of annealed gRNA and 20 μM of Cas9 2NLS (Synthego) were mixed and incubated at room temperature for 10 minutes to make the RNP complex per transfection.

### Transfection of K526 Cells

K562 cells were cultured in RPMI medium containing 10% FBS, and the number of cells was maintained up to 1 million per ml. The cells were passaged a day before transfection. 2.5 million cells were used per transfection. The RNP complex was mixed with 3 μg of the plasmid carrying ADAMTS13-GPI construct and electroporated using a NEPA21 electroporator (Poring pulse setting: Voltage 135, length 2.5 ms, Interval 50 ms, Number of pulse 2, D.rate 10%, Transfer pulse setting: Voltage 20, Length 20 ms, Interval 50 ms, number 5, D. rate 40%). The cells were plated in RPMI medium in a 12-well plate.

The next day the cells were counted and seeded in 96-well plates with density 2 cells and 10 cells per well in medium containing puromycin 2 μg/ml. After 7-10 days puromycin-resistant colonies were picked and expanded.

### Induced-Pluripotent Stem Cells:

iPSCs were reprogrammed from peripheral blood mononuclear cells using the Sendai virus approach (CytoTune-iPS 2.0 Sendai Reprogramming Kit—Thermo Fisher Scientific) according to the manufacturer instruction. Five lines of iPSCs (NY22, OM1, OM2, OM3, and OM4) and three sub-lines of OM1, all generated from healthy controls, were used during these experiments. The vast majority of experiments were performed with lines NY22 and OM1. All lines enucleated at a high rate although the differences might have been associated with different growth rates between lines.

### Pluripotent Stem Cell Culture:

Human pluripotent stem cells (hPSCs) were maintained undifferentiated in E8 medium on Vitronectin (Life Technologies, Carlsbad, Calif.) and passaged using EDTA every 3-4 days depending on their confluence stage (Chen, G., et al. (2011). Nat. Methods 8, 424-429).

Transfection of iPSCs

Undifferentiated iPSCs were passaged 1-2 days before transfection, and 10 μM ROCK inhibitor was added at least an hour before transfection. Cells were harvested and dissociated into single cells using Accutase (Life Technologies, Carlsbad, Calif.) and 2-2.5 million cells were used per transfection reaction. The RNP complex with 3 μg of the plasmid carrying ADAMTS13-GPI construct electroporated using a NEPA21 electroporator (Poring pulse setting: Voltage 125, length 2.5 ms, Interval 50 ms, Number of pulse 2, D.rate 10%, Transfer pulse setting: Voltage 20, Length 20 ms, Interval 50 ms, number 5, D. rate 40%). After electroporation, the cells were plated in 6-well vitronectin coated plates in E8 medium containing 10 uM ROCK inhibitor.

Puromycin selection started 3-4 days after electroporation at concentration 0.3 μg/ml, and resistant colonies were picked and expanded after about one week.

### Screening ADAMTS13-GPI Construct Integration by PCR

Genomic DNA from puromycin-resistant clones was extracted using the WIZARD Genomic DNA Purification kit (Promega, Madison, Wis.). PCRs were performed using Taq PCR Master mix kit (QIAGEN, Hilden, Germany).

Primers for integration detection of the construct at the AAVS1 site

(SEQ ID NO: 14)
P1: GGCCCTGGCCATTGTCACTT
(SEQ ID NO: 15)
P2: GCGTGAGGAAGAGTTCTTG
(SEQ ID NO: 16)
P4: GAGAATCCACCCAAAAGGC

Differentiation of iPSCs into Erythroid Cells: Short PSC-RED Protocol

On day −1: Three-day-old hPSC colonies were dissociated with 5 mM EDTA in PBS for 6 minutes. The EDTA was then removed and replaced with 5 mL of E8 medium, and the well was thoroughly flushed with a 5 mL serological pipet by pipetting up and down 10 times. Small clumps were generated to produce small colonies of about 50 cells on day 0. The cells were then plated at 1-2×10⁵ cells/well in 2 mL/well of E8 medium on vitronectin in tissue culture treated six-well plates (Falcon), which are used throughout the protocol. After plating, the cells were allowed to attach overnight.

On day 0: Differentiation was induced by replacing the E8 medium with IMIT medium, containing supplement 1 (Bone Morphogenic Protein 4 (BMP4) (10 ng/mL), Vascular Endothelium Growth Factor 165 (VEGF) (10 ng/mL), basic Fibroblast Growth Factor (bFGF) (10 ng/mL), Wnt3A (5 ng/mL), Wnt5A (5 ng/mL), Activin A (5 ng/mL) and GSK3β Inhibitor VIII (2 μM) (Olivier et al., 2016))

Before inducing the differentiation, the culture was inspected to ascertain that most of the colonies contained about 50 cells or less. One well of the culture was sacrificed for cell counting in order to calculate the yield of cells at the end of the experiments.

On day 2, 6× concentrated supplement 2 in IMIT was added to each well to bring the final concentration of fresh cytokines to 20 ng/mL of BMP-4, 30 ng/mL of VEGF, 5 ng/mL of Wnt3A, 5 ng/mL of Wnt5A, 5 ng/mL of Activin A, 2 μM of GSK30 Inhibitor VIII, 10 ng/mL of bFGF, 20 ng/mL of SCF and 0.4 ng/mL of beta-Estradiol.

On day 3, the cells were dissociated with TrypleSelect 1× for 5-10 minutes at 37 C. After the addition of 10 mL of PBS, cells were centrifuged for 3 minutes at 250 g, the supernatant was discarded and the cells re-suspended in fresh IMIT medium containing supplement 3 (BMP4 (20 ng/mL), VEGF (30 ng/mL), bFGF (20 ng/mL), SCF (30 ng/mL), Insulin-like Growth Factor 2 (IGF2) (10 ng/mL), Thrombopoietin (TPO) (10 ng/mL), SB431542 (3 μM), Heparin (5 μg/mL), IBMX (50 μM) and beta-Estradiol (0.4 ng/mL) and plated at 1×10⁵ cells/mL of a tissue culture treated six wells plate (3 mL per well).

On day 6, the cells were centrifuged for 3 minutes at 350 g and re-suspended at 5×10⁵/mL in fresh IMIT medium containing supplement 3 without SB431542 but with 30 nM of UM171.

Between day 6 and day 10, the cells were diluted to 0.5×10⁶/mL any time they reached more than 1.5×10⁶ cells/mL by addition of the same medium and supplement. An additional dose of S3 supplement (provided from a 6× concentrated stock) was added at day 8 to fully renew the cytokines and small molecules.

On day 10, the cells were centrifuged for 3 minutes at 250 g, plated at 0.66×10⁵ cells/mL in IMIT containing the SED supplement (SCF 100 ng/mL, Erythropoietin 4 U/mL, IBMX 50 μM and Dexamethasone 1 μM). From day 10 to day 17, the cells were diluted to 0.5×10⁶/mL anytime they reached more than 1.5×10⁶ cells/mL by addition of the same medium and supplement In addition, 6× concentrated SED supplement in IMIT was added every 2 days to fully renew the cytokines and small molecules.

On day 17, the cells were centrifuged for 3 minutes at 250 g and plated at a density of about 2×10⁵/mL of IMIT containing the SER supplement (SCF (50 ng/mL), EPO (4 U/mL) and RU486 (1 μM). From day 17 to 24, the cells were diluted to 0.5×10⁶ any time they reached more than 1.5×10⁶ cells/mL by addition of the same medium and supplement. In addition, 6× concentrated SER supplement in IMIT was added every 2 days to fully renew the cytokines and small molecules.

On day 24, the cells were centrifuged for 3 minutes at 250 g and plated at 2×10⁵/mL in R5 medium with the SER2 supplement (SCF (10 ng/mL), EPO (4 U/mL) and RU486 (1 μM in R5 medium with the SER2 supplement any time they reached more than 1.5×10⁶ cells/mL by addition of the same medium and supplement. In addition, 6× concentrated SER2 supplement in R6 was added every 2 days to fully renew the cytokines and small molecules.

On day 31, the cells were centrifuged for 3 minutes at 250 g and maintained in R5 or R6 medium alone for up to 8 days.

### Long Differentiation Protocol

This long protocol is identical to the short protocol but an additional HPC expansion step in added after day 10. This step consists of centrifuging the cells at 250 g for three minutes and replating the day 10 cells in IMIT at 2×10⁵/mL in the presence of supplement 4 (bFGF (5 ng/mL), SCF (15 ng/mL), VEGF (5 ng/mL), TPO (10 ng/mL), IGF2 (10 ng/mL), Platelet Derived Growth Factor (PDGF) (5 ng/mL), Angiopoietin-like 5 (ANGPTL5) (5 ng/mL), Chemokine Ligand 28 (CCL28) (5 ng/mL), IBMX (30 μM), Heparin (5 μg/mL) and UM171 (30 nM) for one or two weeks. As above the concentration of cells is kept below 1.5×10⁶ cells/mL at all times and cytokines are refreshed every two days by adding 6× concentrated supplement. Cells kept for two weeks in these conditions, were centrifuged and transferred to fresh plates after 7 days to eliminate any attached cells.

After this additional step, the differentiation resumes according to the short protocol day 10. A one-time addition of Granulocyte-Macrophage Colony Stimulating Factor (GM-CSF) (20 ng/mL) and Granulocyte Stimulating Factor (G-CSF) (20 ng/mL) is, however, necessary to induce maximal proliferation of the HPCs in the SED supplements.

### Analysis and Characterization

Cell enumeration: Cells were counted with a Luna-FL dual channel Automated Cell Counter (Logos) using acridine orange to visualize the live cells and propidium iodide to exclude the dead cells

Flow cytometry: iPSCs undergoing differentiation were evaluated by FACS using antibodies against CD34, CD36, CD43, CD45, CD71 and CD235a also known as glycophorin A (BD Biosciences and eBioscience).

Enucleation: The enucleation rate was measured using the DRAQ5 DNA nuclear stain (ThermoFisher) after exclusion of dead cells with Propidium Iodide. The cells were analyzed with a BD FACS Calibur flow cytometer (BD Biosciences) or a DPX10 (Cytek) flow cytometer, and the flow cytometry data were analyzed with the Flowjo software.

Giemsa staining: Erythroid differentiation and enucleation were also assessed microscopically by Rapid Romanovsky staining of cytospin preparations. Cell sizes were estimated on a Nikon TE-2000S microscope using the software provided by the manufacturer.

### Flow Cytometry

Cells were (about 3×10⁵) stained first with mouse monoclonal anti-human ADAMTS13 antibody (Invitrogen, Carlsbad, Calif.) for 20 minutes at 4C, then with a secondary antibody (anti-mouse IgG) conjugated with either FITC or PE (Invitrogen, Carlsbad, Calif.) for 20 minutes.

### FRET Assay

ADAMTS13 activity of the cells was measured using SENSOLYTE 520 ADAMTS13 Activity Assay Kit (AnaSpec, Fremont, Calif.). Cells were harvested and resuspended in 5 ul of PBS in three different concentrations (1×10⁵, 2×10⁵ and 3×10⁵) and added to 5 ul of FRET substrate in 2× assay buffer. Fluorescence was measured at Ex/Em=490/520 nm.

### Example 2

Blood transfusions have been clinically useful for more than 100 years, and the idea that RBCs could be modified to serve as more than just oxygen carriers is almost as old (Villa, C. H. et al. (2016); Adv. Drug Deliv. Rev. 106, 88-103). Drug delivery through therapeutic RBCs as compared to direct injection in the plasma has generated considerable interest because the approach could lengthen the half-life of the therapeutic agent in circulation, spatially restrict the drugs to the lumen of the cardiovascular system which can decrease toxicity by limiting diffusion outside of blood vessels, and shield the drug from the immune system which also decreases the risks of allergy and contributes to increasing half-life as demonstrated by studies on asparaginase encapsulation inside RBCs.

Initial efforts to modify RBCs focused on altering their surface antigens to make them more universal, loading them with various drugs, and decorating them with antibodies or other surface molecules. Despite many technical difficulties, these efforts have been successful, and multiple clinical trials are currently in progress, attesting to the potential of this technology.

RBCs collected from volunteers can be loaded with therapeutically useful content, such as asparaginase or dexamethasone through hypotonic shock. RBCs can also be decorated by attaching proteins to their membrane using, for instance, single chain antibodies targeted to glycophorin A or sortase-catalyzed reaction. These decorated RBCs have been shown to be useful to present antigens, to carry therapeutic drugs, or to immunize against toxins, to cite just a few applications.

As cell culture methods and stem cell biology have progressed, in vitro production of cRBCs has become an alternate strategy to produce RBCs loaded with drugs. One major advantage of in vitro production is that genetically homogeneous cells can be produced from the stem cells of a single rare donor carrying desirable blood groups that are compatible with a very large fraction of the population. If the source cells are immortal, unlimited numbers of cells can be produced, which eliminates the risk of contamination by unknown or emerging pathogens associated with collection of cells from donors, and decreases production complications associated with the genetic heterogeneity of the donors, which are two drawbacks of the use of multiple donors.

Cell Sources for cRBC Production:

Primary cells: Adult primary hematopoietic blood cells were the first human cells expanded and differentiated into cRBCs in liquid culture in vitro. Since then, large progress has been made, and it is now possible to expand the stem and progenitor cells from one unit of cord or peripheral blood into several units of enucleated RBCs. Such yield is theoretically sufficient to expand units of blood with rare phenotypes for small numbers of patients or to generate therapeutic RBCs. However, because this approach does not eliminate the need to collect cells from volunteers, an immortal cell source has been sought. Three types of immortal cells have been considered to produce cRBCs: self-renewing progenitors, immortalized progenitors, and iPSCs.

Immortalized cells: Immortalized animal erythroid progenitors were produced over 30 years ago, but all of the cell lines produced exhibited abnormal karyotypes and enucleated poorly upon terminal differentiation. More recently, more robust immortalization protocols have been developed that yield lines that can terminally differentiate and enucleate at higher but still relatively modest rates. These lines are an exciting avenue of research and may become an important source of cRBCs. However, all lines produced so far are karyotypically unstable, exhibit low growth rate and can only be cultured at low density, in part because of the leakiness of the inducible systems that control the oncogene expression.

iPSCs: Human embryonic stem cells were first differentiated into blood cells in 2001 by Kaufman et al. by co-culture with a feeder layer (Kaufman, D. S. et al. (2001). Proc. Natl. Acad. Sci. 98, 10716-10721). Many investigators, including the inventors, have refined this initial protocol to the point where it became possible to produce thousands of RBCs per iPSC. However, by contrast with the cells produced from adult or cord blood stem and progenitor cells, IPSCs-derived cRBCs exhibited poor enucleation, which had been a major roadblock for the field, which as discussed below were recently resolved by the present disclosure.

IPSCs provide a truly inexhaustible source of cells for industrial production because they are karyotypically stable and easy to produce. Thousands of iPSC lines, which can each be grown for at least 50 passages, can easily be produced from a few milliliters of peripheral blood as a starting material. A drawback of iPSCs, as compared to immortalized progenitors, is that a longer, more complex differentiation protocol is required to produce cRBCs (about 40 days versus 10-20 days). As described below, the scalable protocol as disclosed herein has considerably reduced the complexity of the iPSC differentiation method.

On balance, immortalized progenitors and iPSCs are two exciting sources of cells to produce cRBCs with excellent prospects and both approaches should be pursued. Initially, it has been focused on iPSC differentiation because many of the steps to produced cRBCs are common to both procedures and can be adapted for immortalized progenitors.

TTP, a rare difficult to treat coagulation disorder: TTP is a rare disorder that can be diagnosed by the presence of microangiopathic hemolytic anemia, schistocytes, and thrombocytopenia in the absence of other likely etiologies. (Zheng, X. L. (2015). Annu. Rev. Med. 66, 211-225). Twenty years ago multiple investigators demonstrated that TTP was associated with the presence of ultra-large VWF multimers that was caused by the deficiency of a plasma factor. This plasma factor which was first demonstrated to be a metalloprotease by Tsai et al. (Tsai, H. M. (1996). Blood 87, 4235-4244) and independently by Furlan et al. (Furlan, M., et al. (1998). Blood 91, 2839-2846) was eventually sequenced and identified as ADAMTS13. Positional cloning demonstrated that ADAMTS13 was also responsible for the congenital form of TTP.

Some of the molecular aspects of TTP are now well understood. Low ADAMTS13 activity decreases the normal cleavage rate of Von Willebrand factor (VWF), which therefore accumulates in its high molecular weight form. These high molecular weight VWF molecules, unfold in the presence of shear stress in the circulation and interact with the vessel walls and platelets promoting thrombi formation in the absence of injury, which can lead to life-threatening microvascular thrombosis and the clinical manifestations of TTP.

Auto-antibodies: The idiopathic form of TTP has an incidence of about 1/250,000 per year and is caused by auto-antibodies that inactivate ADAMTS13. Anti-ADAMTS13 antibodies are mostly IgG4 and IgG1 and can either inhibit the proteolytic activity, enhance the clearance, or disturb the interaction with physiologic binding partners of ADAMTS13. Importantly, epitope mapping revealed that in more than 80% of the patients, these antibodies recognized regions of ADAMTS13 that are outside of the catalytic site, which suggested that it might be possible to develop forms of ADAMTS13 that remain catalytically active but that are not inhibited by the most common auto-antibodies.

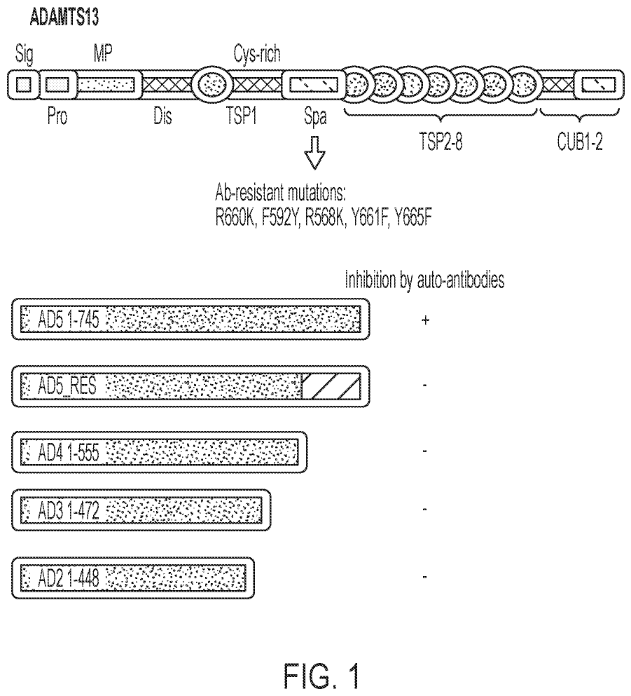

Indeed, there are several variants retaining significant VWF cleaving activity but not inhibited by patients auto-antibodies (FIG. 1). More recently, Jian et al. genetically engineered full-length ADAMTS13 containing amino-acid changes that confer resistance to some of the most common auto-antibodies (Jian, C., et al. (2012). Blood 119, 3836-3843) (FIG. 1). These important observations led the way to the production of therapeutic products that are resistant to the auto-antibodies which are responsible for idiopathic TTP.

This is important because the current treatment for idiopathic TTP relies on plasma exchange requiring infusion of 2 to 4 liters of concentrate for up to several weeks. Plasma exchange, complemented or not with rituximab, an anti-CD20 Ab that suppresses the production of autoantibodies, or with Caplacizumab, a nanobody of VWF that blocks VWF-platelet aggregation, is a life-saving but cumbersome procedure that has significant toxicity (mostly allergies), a high number of relapses, and a 10-20% rate of mortality.

Congenital TTP represents about 5% of all TTP cases. The penetrance is very high (90%), but the age of onset and the severity and frequency of the episodes varies between patients, because most affected individuals are compound heterozygous and exhibit variable levels of residual ADAMTS13 activity. Congenital TTP is treated in a similar manner as the idiopathic form but with lower doses of plasma.

Recombinant ADAMTS13 is currently being tested to treat congenital TTP. This approach could also potentially be used to treat the idiopathic form. However, in the absence of an antibody resistant form of recombinant ADAMTS13 with a long half-life, infusion of very large amounts of the proteins will be required in order to saturate the auto-antibodies, since plasma exchange works in large part by removing the auto-antibodies.

Animal models of TTP: Shortly after the cloning of ADAMTS13, germline knock-outs in C57/B16 or 129/Sv mice were generated but exhibited normal survival and only a mild VWF-platelet interaction phenotype. However, Desch et al. obtained a phenotype quite similar to human congenital TTP in the CASA genetic background and demonstrated that in this genetic background, a TTP-like disease could be triggered by injection of Shiga-toxin (Desch, K. C., et al. Vasc. Biol. 27, 1901-1908; Motto, D. G., et al. (2005) J. Clin. Invest. 115, 2752-2761). Although the levels of VWF are higher in CASA mice, additional analysis revealed that unidentified additional genetic factors were mostly responsible for the TTP phenotype in these mice. More recently Schivitz et al. developed an alternate model in which TTP-like symptoms are induced in the ADAMTS13^{KO} by injection of recombinant VWF (Schiviz, A., et al. (2012). Blood 119, 6128-6135). This model, which results in thrombocytopenia, schisocyte formation, anemia, weight loss, high LDH and histological evidence of thrombosis, is experimentally more tractable because the phenotype is detectable in the C57/B16 background and is not dependent on unidentified genetic factors.

Acquired TTP has been modeled in wild-type mice by injection of rabbit polyclonal anti-ADAMTS13 antibodies, and in baboons using monoclonal antibodies.

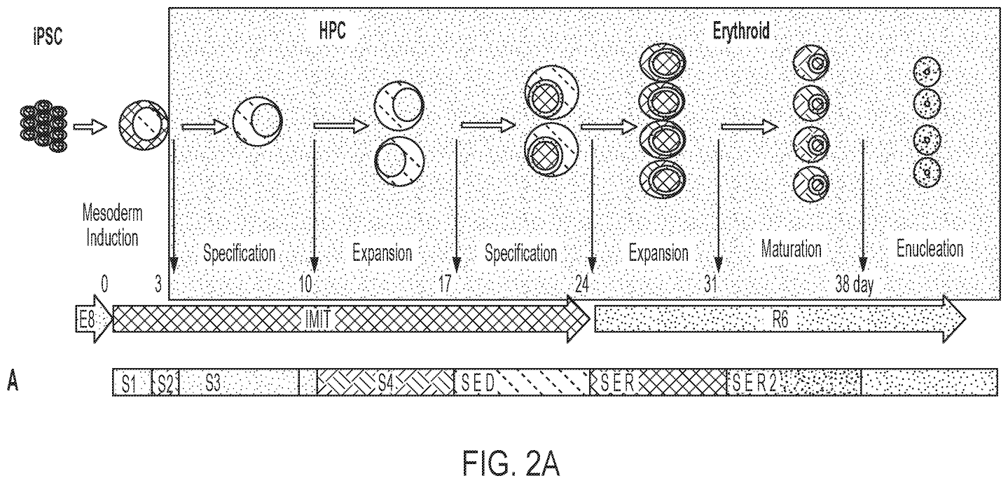

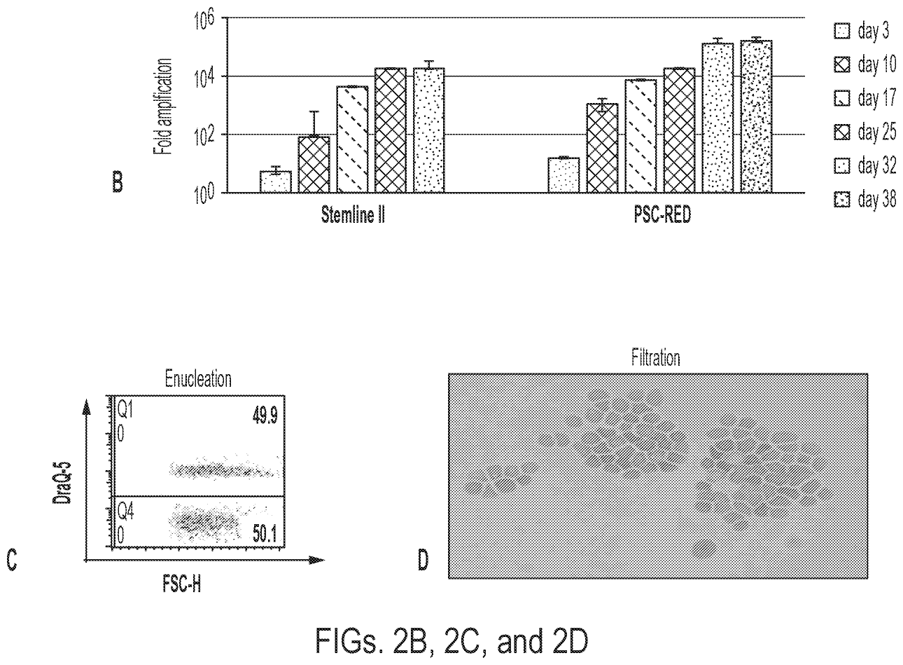

PSC-RED is a chemically-defined method to produce cRBCs: This disclosure provides a chemically defined, albumin-free robust Pluripotent Stem Cell Erythroid Differentiation (PSC-RED) protocol to produce enucleated cRBCs from human iPSCs (FIGS. 2A and 2B). PSC-RED is associated with a rate of enucleation averaging 50% and reaching 75% in the best experiments (FIGS. 2C and 2D), presumably because undefined animal-derived contaminants inhibited enucleation in previous protocols.

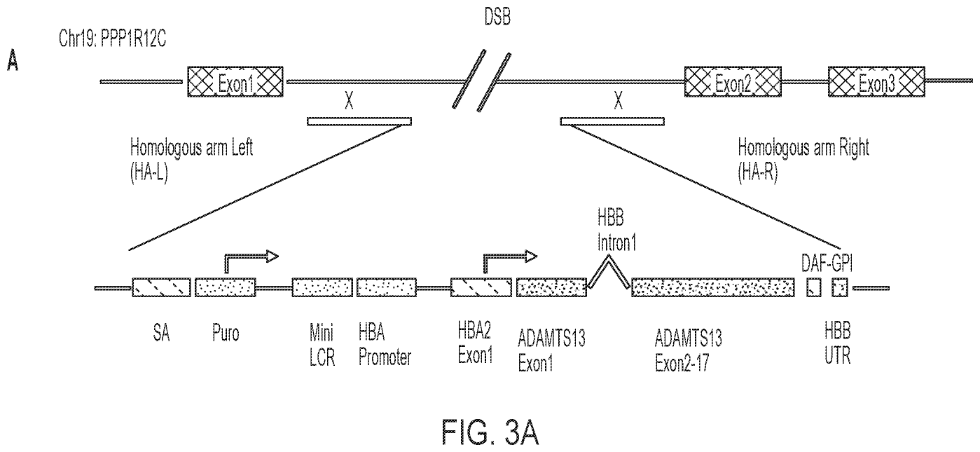

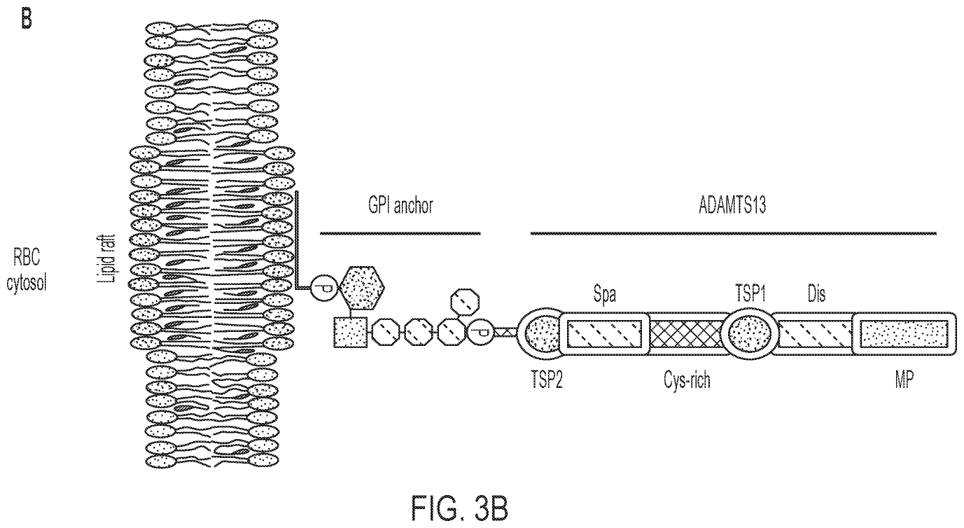

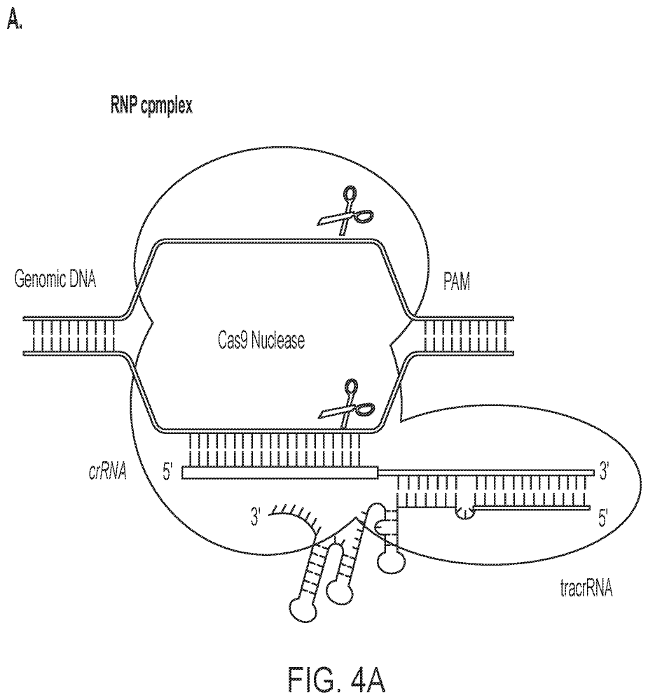

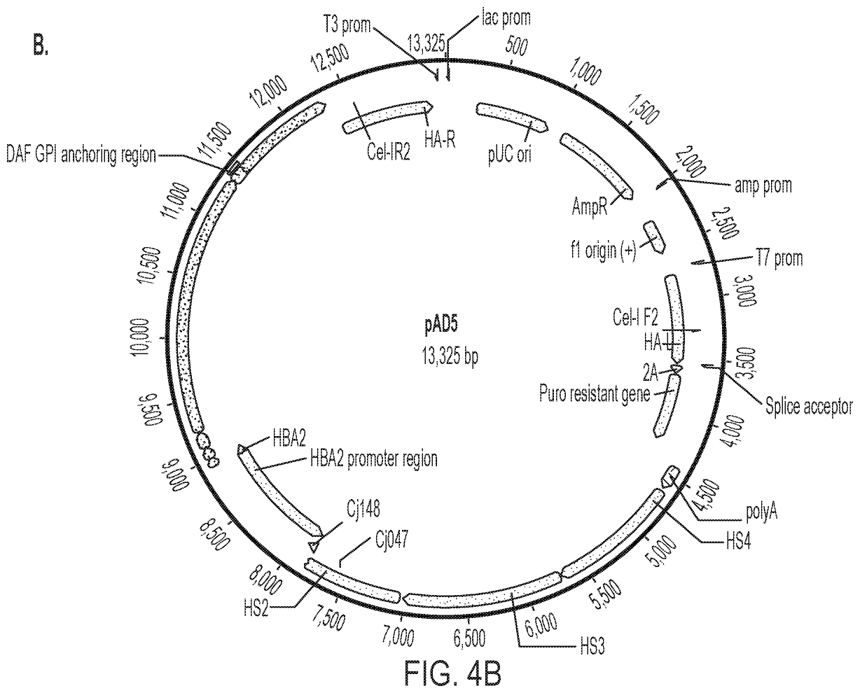

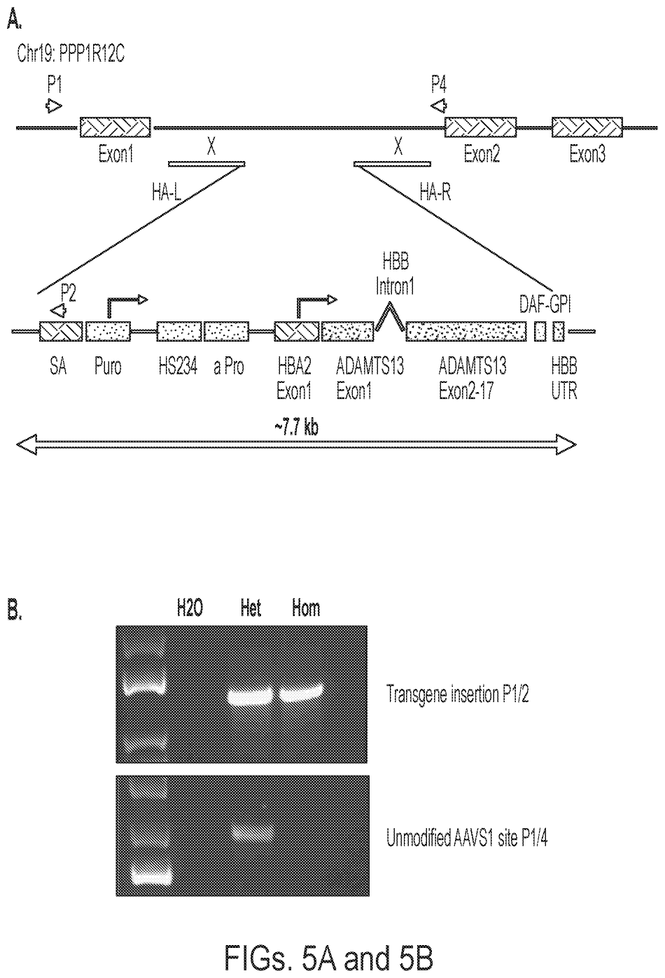

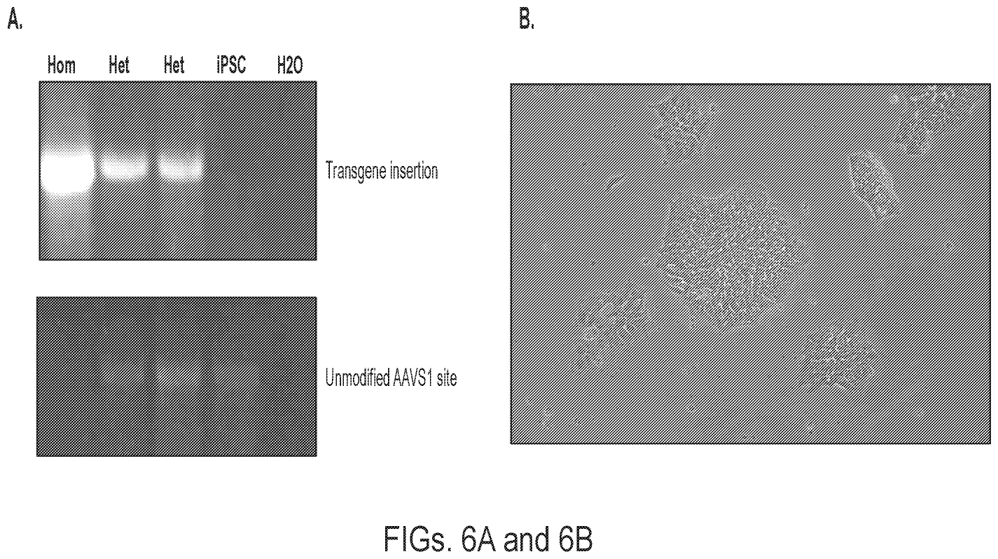

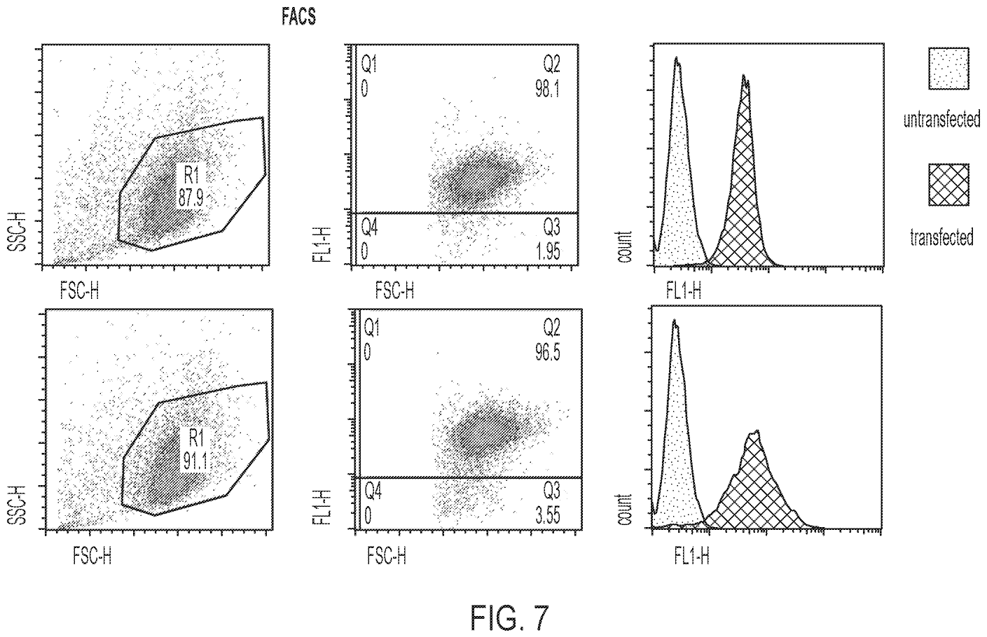

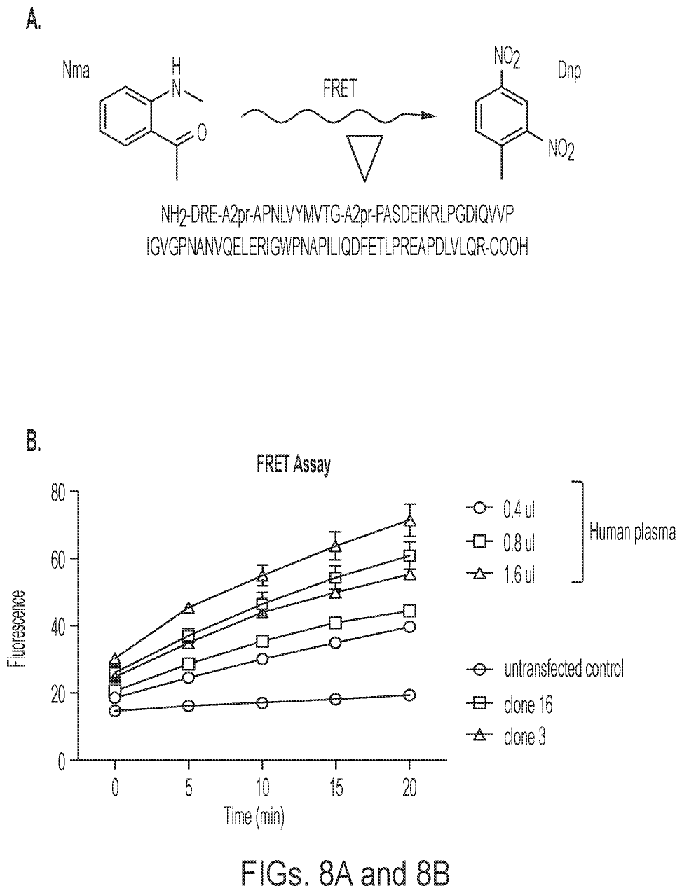

ADAMTS13-cRBCs: To determine if it was possible to express a functionally active membrane-targeted form of ADAMTS13, GPI-AD5, a fusion construct, were generated, in which AD5 (FIG. 3), a 700 amino-acid N-terminal fragment of ADAMTS13 was fused to a GPI-anchor peptide derived from the DAF gene. Transgenes in iPSCs was expressed in an erythroid-specific manner. The fusion constructs 3′ of the alpha-globin promoter, and the mini-LCR was cloned and inserted at the AASV1 safe harbor in K562 cells using a CRISPR cas9 system (FIGS. 3, 4 and 5). The same construct was also inserted at the AAVS1 site in human iPSCs using the same method (FIG. 6). FACS analysis using an anti-ADAMTS13 antibody revealed that GPI-AD5 was expressed at high levels on the membrane of K562 cells (FIG. 7). FRET analysis using the VWF73 FRET assay, in which a peptide containing the recognition site of ADAMTS13 in the VWF is cleaved, demonstrated that it was enzymatically active (FIG. 8). Comparison with ADAMTS13 activity in plasma revealed that expression was very high since 200,000 ADAMTS13 expressing cells provided about the same activity had about the same activity GPI-ADAMTS13 as 10 μL of plasma, which suggests that transfusion of about 5 mL of cRBCs would be sufficient to provide the same ADAMTS13 activity as one liter of plasma.

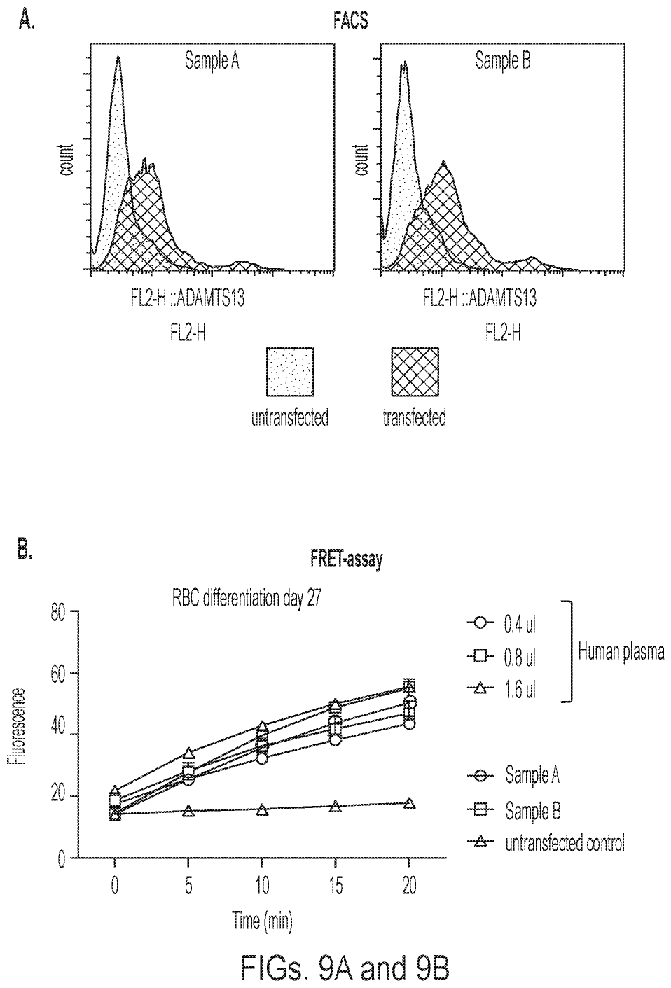

Analysis of erythroid cells derived from GPI-ADAMTS13 iPSC revealed that as the K562 cells, they expressed GPI-ADAMTS13 (AD5) on their membrane (FIG. 9A), and that the fragment was enzymatically active (FIG. 9B).

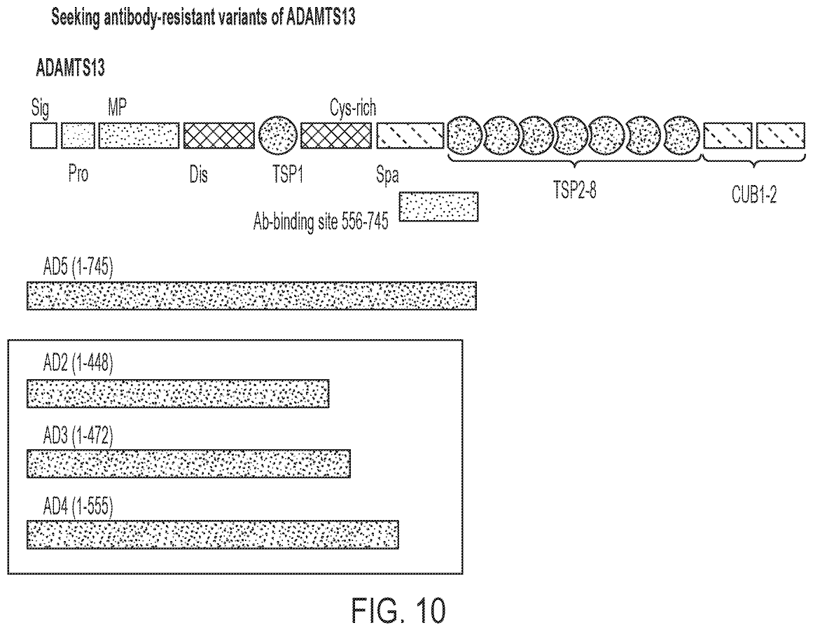

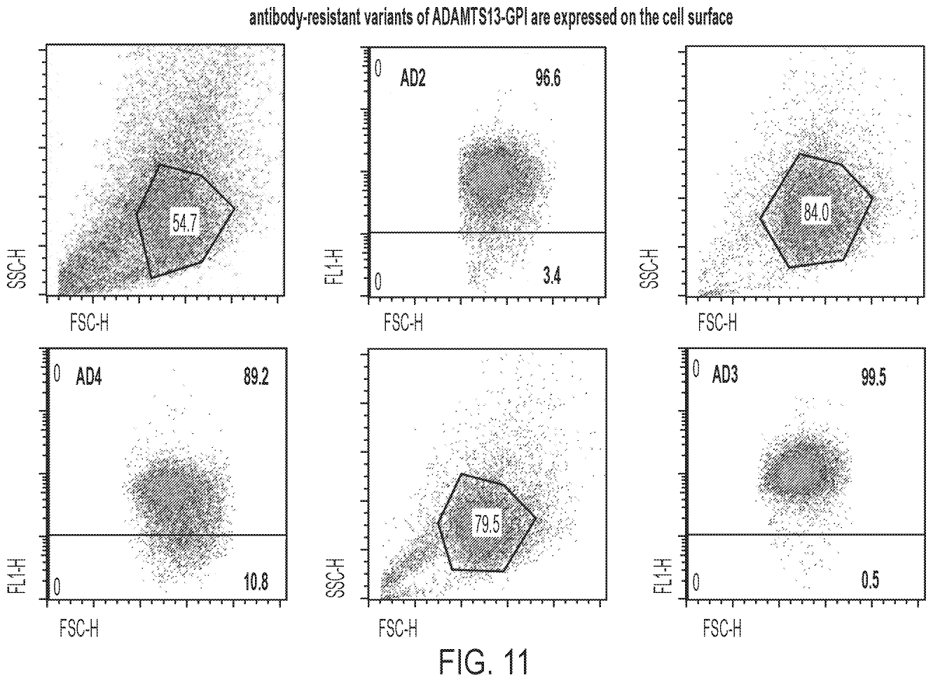

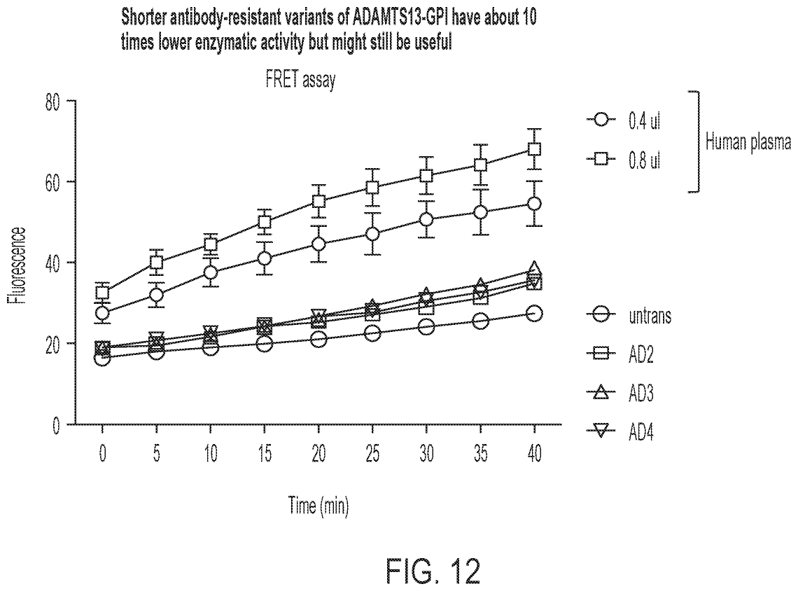

To determine if the same approach can be used to generate cells that would express the antibody-resistant form of ADAMTS13, constructs AD2, AD3, and AD4 were generated, which contains three truncated variants forms of ADAMTS13 fused to the GPI anchor, which had previously been shown to be resistant to TTP inhibitors (FIG. 10). These constructs were then transfected in K562 and expression of the truncated form of GPI-ADMTS13 was assessed by flow cytometry. This revealed that these truncated form of GPI-ADAMTS13 could be expressed at high levels on the membrane of K562 cells (FIG. 11). Analysis of the K562 cells expressing the truncated GPI-ADAMTS13 using the VWF74 FRET assay demonstrated that all three constructs were enzymatically active, although at lower levels than the AD5 construct (FIG. 12).

Together this data demonstrates that is possible to express on the membrane of erythroid cells, a truncated variant of ADAMTS13 that are sensitive or resistant to the antibodies that are responsible for TTP (TTP inhibitors) and enzymatically active.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

1. A genetically modified red blood cell, wherein the red blood cell is engineered to express on the surface thereof a fusion protein comprising a fragment of ADAMTS13 that is enzymatically active against von Willebrand factor (VWF).

2. The red blood cell of claim 1, wherein the fragment of ADAMTS13 has an amino acid sequence at least 75% identical to the sequence of SEQ ID NOs: 1, 2, 3, 8, 9, 10, 11, 12, or 13.

3. The red blood cell of claim 1, wherein the fusion protein comprises a lipid anchor operably linked to the fragment of ADAMTS13.

4. The red blood cell of claim 1, wherein the lipid anchor is a Glycosylphosphatidylinositol (GPI) anchor.

5. The red blood cell of claim 4, wherein the GPI anchor comprises the amino acid sequence of SEQ ID NO: 4.

6. The red blood cell of claim 1, wherein the red blood cell is transduced with a retrovirus comprising a nucleic acid encoding the fusion protein.

7. The red blood cell of claim 6, wherein the nucleic acid comprises a nucleotide sequence at least 75% identical to a nucleic acid sequence of SEQ ID NOs: 5-7.

8. A method for treating thrombotic thrombocytopenic purpura (TTP), comprising administering a therapeutically effective amount of the genetically modified red blood cells of claim 1 to a subject in need thereof.

9. The method of claim 8, wherein TTP is hereditary TTP, congenital TTP, acquired TTP, or immune-mediated TTP.

10. The method of claim 8, wherein the red blood cells are administered by infusion.

11. The method of claim 8, comprising producing the red blood cells in vitro before administering to the subject.

12. The method of claim 11, wherein the red blood cells are produced in a hollow fiber culturing system by expansion of hematopoietic progenitors.

13. A method of preparing the red blood cell of claim 1, comprising:
(i) providing a plurality of stem cells;
(ii) contacting the stem cells with a nucleic acid encoding a fusion protein comprising ADAMTS13 or fragment thereof to obtain transduced stem cells;
(iii) expanding the transduced stem cells in cell culture medium; and
(iv) collecting resulting red blood cells.

14. A composition comprising the red blood cells of claim 1 and optionally a cryo-protectant.

15. Blood, cellular and acellular blood components, or blood products obtained from the red blood cell of claim 1.

16. A method for increasing a level of functional ADAMTS13 in a subject, comprising administering an effective amount of the red blood cells of claim 1 to the subject.

17. A method for decreasing aggregation of VWR in a subject, comprising administering an effective amount of the red blood cells of claim 1 to the subject.


SEQUENCE LISTING (16 sequences)

SEQ ID NO: 1 - protein, 1427 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 2 - protein, 745 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPY

SEQ ID NO: 3 - protein, 782 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYPNKGSGTTSGTTRLLSGHTCFTLTGLLGTLVTMGLLT

SEQ ID NO: 4 - protein, 37 aa, Homo sapiens
PNKGSGTTSGTTRLLSGHTCFTLTGLLGTLVTMGLLT

SEQ ID NO: 5 - DNA, 4284 bp, Homo sapiens
atgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggtggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtacgcccagcctggtgccccacgaagaagccgctgctccaggacggaccacagccacccctgctggtgcctccctggagtggtcccaggcccggggcctgctcttctccccggctccccagcctcggcggctcctgcccgggccccaggaaaactcagtgcagtccagtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctga

SEQ ID NO: 6 - DNA, 2235 bp, Homo sapiens
atgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctac

SEQ ID NO: 7 - DNA, 2346 bp, Homo sapiens
atgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctacccaaataaaggaagtggaaccacttcaggtactacccgtcttctatctgggcacacgtgtttcacgttgacaggtttgcttgggacgctagtaaccatgggcttgctgact

SEQ ID NO: 8 - DNA, 111 bp, Homo sapiens
ccaaataaaggaagtggaaccacttcaggtactacccgtcttctatctgggcacacgtgtttcacgttgacaggtttgcttgggacgctagtaaccatgggcttgctgact

SEQ ID NO: 9 - protein, 1353 aa, Homo sapiens
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 10 - protein, 1371 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 11 - protein, 1297 aa, Homo sapiens
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 12 - protein, 1340 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 13 - protein, 1266 aa, Homo sapiens
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 14 - DNA, 20 bp, Artificial
  Synthetic
ggccctggccattgtcactt

SEQ ID NO: 15 - DNA, 19 bp, Artificial
  Synthetic
gcgtgaggaagagttcttg

SEQ ID NO: 16 - DNA, 19 bp, Artificial
  Synthetic
gagaatccacccaaaaggc